(19) United States
(12) Patent Application Publication
(10) Pub. No.: US 2024/0262878 A1
(43) Pub. Date: Aug. 8, 2024

(54) PHOTOACTIVATABLE GASDERMIN PROTEINS

(71) Applicant: THE BOARD OF TRUSTEES OF THE UNIVERSITY OF ILLINOIS, Chicago, IL (US)

(72) Inventor: Chia Hao MO, Chicago, IL (US)

(21) Appl. No.: 18/567,159

(22) PCT Filed: Jun. 7, 2022
(86) PCT No.: PCT/US2022/032476

§ 371 Date: Dec. 5, 2023

Related U.S. Application Data
(60) Provisional application No. 63/197,672, filed on Jun. 7, 2021.

(51) Int. Cl. C07K 14/47; A61K 41/00; C12N 13/00

(52) U.S. Cl. CPC C07K 14/4747; A61K 41/00; C12N 13/00; C07K 2319/70

(57) ABSTRACT

This invention relates to a modified gasdermin protein having a photoactivatable linker inserted between the C-terminal domain and N-terminal domain a gasdermin protein, nucleic acids and vectors encoding the modified gasdermin protein, and methods of using the modified gasdermin protein to introduce agents into cells and facilitate the treatment of diseases or conditions.

This application claims benefit of priority to U.S. Provisional Patent Application Ser. No. 63/197,672, filed Jun. 7, 2021, the content of which is incorporated herein by reference in its entirety.

This invention was made with government support under grant nos. HL060678, HL077806 and HL090152 awarded by the National Institutes of Health. The government has certain rights in this invention.

### BACKGROUND

Programmed cell death plays an important role in the growth, development, and homeostasis of multicellular organisms. Pyroptosis is a lytic cell death program triggered by inflammatory caspase-11 in mice and caspase-1/4/5 in humans upon both canonical and non-canonical inflammasome activation. These caspases cleave gasdermins, whose N-terminal domain then inserts into the bilayer membrane and forms oligomeric pores. The most extensively studied gasdermin, the 53 kDa Gasdermin-D (GSDMD), is an essential mediator of pyroptosis in human and murine cells (He et al. (2015) Cell Res. 25:1285-1298; Broz et al. (2020) Nat. Rev. Immunol. 20:143-157; Shi et al. (2015) Nature 526:660-665; Sato et al. (1998) Mamm. Genome 9:20-25). GSDMD forms ˜21 nm diameter pores (Liu et al. (2016) Nature 535:153-158) in the plasmalemmal membrane (Shi et al. (2015) Nature 526:660-665), causing osmotic imbalance, cell volume instability, and cell lysis. In T-cells and macrophages, GSDMD pores affect immune functions through the release of pro-inflammatory cytokines such as IL-Iβ and IL-18 into the cellular milieu (Place (2019) J. Exp. Med. 216: 1474-1486). It has also been shown that GSDMD-driven pyroptotic endothelial cell death in blood vessels is a significant driver in inflammatory lung injury (Cheng et al. (2017) J. Clin. Invest. 127: 4124-4135).

In contrast to apoptosis, where the cell death program is irreversible once set in motion, gasdermin-mediated pyroptosis is separable from cell death (DiPeso et al. (2017) Cell Death Discov. 3:17070). This is also reflected in immune cell priming, where only a fraction of caspase-active cells undergoes catastrophic cell death and many retain a molecular memory of the activation. While GSDMD pores are removed on a long time scale through exosomal scission (Rühl et al. (2018) Science 362:956-960), their large size makes them highly damaging to homeostasis they cellular should remain permanently open until removal. Thus, apart from irreversible caspase activation, gasdermin pores must rely on a novel mechanism to separate activation from lysis. Despite their importance, little is known about the dynamic conformations of GSDMD pores and how they interact with cell signaling.

### SUMMARY OF THE INVENTION

This invention is a modified gasdermin protein composed of a photoactivatable linker inserted between the N-terminal domain and C-terminal domain of a gasdermin protein (e.g., gasdermin A, gasdermin B, gasdermin C, gasdermin D, gasdermin E, pejvakin, or a fragment thereof), wherein the photoactivatable linker dimerizes or dissociates upon illumination. In some aspects, the photoactivatable linker is inserted at an endogenous protease cleavage site of the gasdermin protein. In other aspects, the photoactivatable linker is an optogenetic dimerization protein selected from the group of Vivid, cryptochrome, N-terminal domain of cryptochrome-interacting basic-helix-loop-helix protein 1, phytochrome, phytochrome interacting factor, UV-B photoreceptor, Flavin-binding Kelch repeat F-box 1, GIGANTEA, TULIPS, Dronpa, iLID, AsLOV variant and combinations thereof. In further aspects, the photoactivatable linker is a photocleavable protein that dissociates into at least two fragments or releases one end of a loop insertion upon illumination, e.g., PhoCle0.1, PhoCle0.2, PhoCle0.3, PhoCle0.4, PhoCle0.5, PhoCle0.6, PhoCle0.7, cpPhoCle, PhoCl2c PhoCl2f. In particular aspects, the photocleavable protein has the amino acid sequence of SEQ ID NO: 23, SEQ ID NO: 24, SEQ ID NO: 25, SEQ ID NO:26, SEQ ID NO: 27, SEQ ID NO: 28, SEQ ID NO: 29, SEQ ID NO: 30, or SEQ ID NO: 31 and is optionally cleaved by illumination with light having a wavelength of about 400 nm to 450 nm. In specific aspects, the modified gasdermin protein has the amino acid sequence of SEQ ID NO:33, SEQ ID NO: 34, SEQ ID NO:35, SEQ ID NO: 36, SEQ ID NO: 37, SEQ ID NO: 38, SEQ ID NO: 39 or SEQ ID NO: 40. A recombinant nucleic acid encoding the modified gasdermin protein, a recombinant vector including said nucleic acid, and a recombinant host cell harboring the modified gasdermin protein are also provided.

In one aspect, this invention provides a method of modulating gasdermin pore formation in a cell by inserting a photoactivatable linker between the N-terminal domain and C-terminal domain of a gasdermin protein of a cell and illuminating the cell with light having a wavelength suitable to activate the photoactivatable linker.

In another aspect, this invention provides a method of treating a disease or condition in a subject wherein gasdermin pore formation in a cell of the subject confers a benefit, wherein the method includes the steps of inserting a photoactivatable linker between the N-terminal domain and C-terminal domain of a gasdermin protein of a cell and illuminating the cell with light having a wavelength suitable to activate the photoactivatable linker, thereby modulating gasdermin pore formation in the subject and treating the disease or condition.

In a further aspect, this invention provides a method of facilitating transport of a therapeutic agent into a cell by inserting a photoactivatable linker between the N-terminal domain and C-terminal domain of a gasdermin protein of a cell; contacting the cell with a therapeutic agent; and illuminating the cell with light having a wavelength suitable to activate the photoactivatable linker and modulate gasdermin pore formation thereby facilitating transport of the therapeutic agent into the cell.

In yet a further aspect, this invention provides a method of facilitating treatment of a disease or condition in a subject, wherein the method includes the steps of inserting a photoactivatable linker between the N-terminal domain and C-terminal domain of a gasdermin protein of a subject; administering to the subject a therapeutic agent for treating a disease or condition; and exposing the subject to light having a wavelength suitable to activate the photoactivatable linker and modulate gasdermin pore formation so that transport of the therapeutic agent is enhanced and treatment of the disease or condition is facilitated.

### BRIEF DESCRIPTION OF THE DRAWINGS

FIG. 1 illustrates the domain structure and scheme for the optogenetically activatable GSDMD, referred to herein as “PhoDer.” C-terminal autoinhibition is cleaved after blue light exposure, liberating the N-terminal pore domain for membrane insertion.

FIG. 2 shows that intracellular calcium progression in representative cells over a 1-hour optogenetic activation time course could be categorized into the flare, saturation, and leakage phases.

FIG. 3 shows a schematic for the electrophysiological setup used to monitor reconstituted GSDMD pores in a planar lipid bilayer in vitro. Silver-silver chloride electrodes connecting cis and trans compartments conducted the current, which was subsequently amplified and recorded.

FIG. 4 shows representative traces of GSDMD protein-membrane interaction and single pore formation events in lipid bilayers using a gap-free protocol. Recordings in controls including baseline bilayer alone (POPE/POPC bilayer only), baseline bilayer with the addition of GSDMD protein only, and baseline bilayer with the addition of caspase-1 only, were absent of current activity. By comparison, addition of activated GSDMD (GSDMD+caspase-1) to the baseline bilayer demonstrated protein-membrane interaction characterized by fluctuating micro-currents.

FIG. 5 shows that PI(4,5)P₂ (PIP2) and PI(3,4,5)P₃ (PIP3) content significantly shortened the time to observe GSDMD protein-membrane interaction compared to baseline phospholipid bilayer. The reported statistical significance was derived from one-way ANOVA with multiple comparisons; “ref” represents the reference; ****p<0.0001.

FIG. 6 shows a histogram of single pore open-close events in different bilayer compositions, wherein either phosphoinositide significantly reduced the current. The reported statistical significance was derived from one-way ANOVA with multiple comparisons; “ref” represents the reference; ****p<0.0001.

FIG. 7 shows that the dwell time (open duration) of GSDMD pores is similar between PE/PC and PIP2-containing bilayers; PIP3-containing bilayers induced apparently short dwell time but bilayer suffered strong osmotic pressure because pores could not close. The reported statistical significance was derived from one-way ANOVA with multiple comparisons; “ref” represents the reference; ****p<0.0001; ***p=0.0002.

FIG. 8 shows significant changes in pore activity were generated by altering the charge or hydrogen bond potential at the indicated sites. Mutations were made in PhoDer at the corresponding GSDMD N-terminal sites. Mutant labels: m1: K51Q/R53Q/K55Q; m2: K51E/R53E/K55E; m3: R42Q/K430; and m5: R42Q/K43Q/K51Q/R53Q/K55Q. N numbers for each mutant are marked below the violin plots. Statistical significance was reported from two-way ANOVA with Tukey's multiple comparisons, ****p<0.0001.

FIG. 9 illustrate pixel-wise calcium fluctuations showing pore dynamics changes with phosphoinositide modulation. Statistical significance was reported from individual paired f- and t-tests with two-tailed Welch's correction; “ref” represents the reference, ***p=0.0003 (no-pretreat vs. wortmannin (“wort”); ****p<0.0001 (no-pretreat vs. U73122 and no-Ca²⁺),

FIG. 10 shows that IL-1B release, induced by short-term LPS stimulation, was dampened by wortmannin but enhanced by U73122. Data were obtained from ELISA measurements over a number of technical replicates (tr) of bone marrow-derived macrophages (BMDM) from separate mice (n). n=3, tr=8. Statistical significance was reported from Brown-Forsythe and Welch ANOVA with multiple comparisons; “ref” represents the reference; in all instances ****p<0.0001; **p=0.0011; ns not significant.

FIG. 11 shows that diacylglycerol (DAG)-rich liposomes dampen IL-1β induced short-term LPS stimulation. Data were obtained from ELISA measurements over a number of technical replicates (tr) of bone marrow-derived macrophages (BMDM) from separate mice (n). n=3, tr=8. Statistical significance was reported from Brown-Forsythe and Welch ANOVA with multiple comparisons; “ref” represents the reference; in all instances ****p<0.0001; ns not significant.

FIG. 12 shows that optogenetic activation of PhoDer delivers actin labels into living cells in a highly significant manner compared to control case where PhoDer was not used and thus no actin was labeled.

FIG. 13 shows that reversibility of an optogenetic gasdermin variant (SEQ ID NO:40) is achieved compared to natural pyroptosis, which is irreversible. Cells were subjected to activations every 20 cycles and their response was expressed by autocorrelation function. The results showed significant rises in multiples of 20 (20, 40, etc.) thereby indicating the cyclic nature of this optogenetic gasdermin variant.

FIG. 14 shows that isoproterenol (ISO) stimulation of beta2-adrengeric receptor activity, as determined by protein kinase A activity, is inhibited by a beta2-adrengeric receptor nanobody delivered into the cytosol by PhoDer.

FIG. 15 shows higher levels of expression of PhoDer correlate with higher levels of beta2-adrengeric receptor inhibition (as determined by protein kinase A activity) with a beta2-adrengeric receptor nanobody delivered into the cytosol.

### DETAILED DESCRIPTION OF THE INVENTION

This invention relates to a modified gasdermin protein composed of a photoactivatable linker inserted between the C-terminal domain and N-terminal domain of a gasdermin protein, as well as nucleic acids encoding the modified gasdermin protein and methods of using the same to control pore size in a cell and treat a disease or condition.

As used herein, the term “gasdermin” refers to a member of the gasdermin family of proteins or polypeptides that can oligomerize and assemble into a membrane pore. In humans, members of the gasdermin family include, but are not limited to, gasdermin A, gasdermin B, gasdermin C, gasdermin D, GSDME (also known as DENA5), PJVK (also known as DENB59 or pejvakin) (Zou et al. (2021) Front. Immunol. 12:751533). In addition to human gasdermin, the invention also includes the orthologs of human gasdermin proteins or polypeptides isolated from primates, cats, dogs, swine, cattle, sheep, goats, horses, rabbits, rats, and mice, as well as fungi such as Podospora anserina (Clave et al. (2022) Proc. Natl. Acad. Sci. USA 119(7):e2109418119) and bacteria (Johnson et al. (2022) Science 375 (6577):221-225). The amino acid sequences of gasdermin family members are provided in Table 1.

TABLE 1

| GSDM | Source | Accession No. | SEQ ID NO: |
|---|---|---|---|
| A | Homo sapiens | NP_835465.2 | 1 |
| | Mus musculus | NP_001007462.1 | 2 |
| | Rattus norvegicus | NP_001101767.1 | 3 |
| B | Homo sapiens | Q8TAX9.2 | 4 |
| | Bos taurus | NP_001231146.1 | 5 |
| C | Homo sapiens | NP_113603.1 | 6 |
| | Mus musculus | NP_113555.1 | 7 |
| | Rattus norvegicus | NP_001127967.1 | 8 |
| D | Homo sapiens | NP_001159709.1 | 9 |
| | Mus musculus | NP_081236.1 | 10 |
| E | Homo sapiens | NP_004394.1 | 11 |
| | Mus musculus | NP_061239.1 | 12 |
| | Rattus norvegicus | NP_001178678.1 | 13 |
| PJVK | Homo sapiens | NP_001036167.1 | 14 |
| | Bos taurus | NP_001180112.1 | 15 |
| | Mus musculus | NP_001074180.1 | 16 |

In certain aspects, the gasdermin protein of this invention is a human gasdermin protein. In other aspects, the gasdermin protein of this invention is selected from gasdermin A, gasdermin B, gasdermin C and gasdermin D. In further aspects, the gasdermin protein is selected from human gasdermin D (hGSDMD), human gasdermin B (hGSDMB) and mouse gasdermin A3 (mGSDMA3). In yet further aspects, the gasdermin protein is selected from human gasdermin D (hGSDMD) and human gasdermin B (hGSDMB). “Gasdermin” as used herein also includes any fragment of a member of the gasdermin family of proteins or polypeptides, or any fragment of an ortholog of a member of the gasdermin family of proteins or polypeptides, wherein said fragment retains at least one biological function that is of interest in the present context.

Structurally, the gasdermin family members contain an N-terminal domain (NTD), also referred to as a lytic domain or membrane domain, which is capable of forming membrane pores to induce cytolysis. In addition, the gasdermin family members contain a C-terminal domain (CTD), also referred to as a repressor domain or autoinhibitory domain, which functions to inhibit cell killing through h intramolecular domain association (Aglietti et al. (2016) Proc. Natl. Acad. Sci. USA 113:7858-7863; Chen et al. (2016) Cell Res. 26:1007-1020; Ding et al. (2016) Nature 535:111-116; Liu et al. (2016) Nature 535:153-158; Rogers et al. (2017) Nat. Commun. 8:14128; Sborgi et al. (2016) EMBO J. 35:1766-1778; Wang et al. (2017) Nature 547:99-103). Between the NTD and CTD is a linker domain, which generally includes a protease cleavage site for granzyme A or caspases such as caspase-1, caspase-3, caspase-8, caspase-11 and the like. Exons encoding the NTD of all human gasdermin genes are conserved (Angosto-Bazarra et al. (2022) BMC Biology 20:9). These exons encode the main secondary structural features of the NTD, i.e., the initial α-helix is encoded by exon II, β-sheets 1 and 2 in exon III, and β-sheets 3 and 4 in exons IV and V. By comparison, exons encoding the CTD and linker domain are less conserved (Table 2).

TABLE 2

| | Exons | | Exons |
|---|---|---|---|
| | Encoding | Exons Encoding | Encoding |
| hGSDM | NTD | Linker Domain | CTD |
| A | I-V | VI-VIII | IX-XII |
| B | I-V | VI | VII-X |
| C | I-V | VI-X | XI-XIV |
| D | I-V | VI-VII | VIII-XI |
| E | I-V | VI | VII-X |
| PJVK | I-V | VI | VII |

The modified gasdermin proteins of the invention include a photoactivatable linker. In particular aspects, the photoactivatable linker is inserted into the modified gasdermin such that the pore-forming capacity of the gasdermin remains substantially the same. In some aspects, the photoactivable linker is inserted between the NTD and CTD of the gasdermin. In other aspects, the photoactivable linker is inserted between the NTD and CTD of human gasdermin A, human gasdermin B, human gasdermin C, human gasdermin D, human gasdermin E, human pejvakin, or a fragment or ortholog thereof. In further aspects, the photoactivable linker is inserted into the linker domain of the gasdermin. In further aspects, the photoactivable linker is inserted at an amino acid encoded by an exon downstream of exon 5. In yet other aspects, the photoactivable linker is inserted at an amino acid encoded by an exon downstream of exon 5 of human gasdermin A, human gasdermin B, human gasdermin C, human gasdermin D, human gasdermin E, human pejvakin, or a fragment or ortholog thereof. In particular aspects, the photoactivable linker is inserted at an amino acid encoded by exon VI, VII or VIII of human gasdermin A; exon VI of human gasdermin B; exon VI, VII, VIII, IX or X of human gasdermin C, exon VI or VII of human gasdermin D, exon VI of human gasdermin E, or exon VI of human pejvakin. In certain aspects, the photocleavable linker is inserted at an amino acid residue located between P242 and E285 in hGSDMD (SEQ ID NO: 9), between D225 and R247 in hGSDMB (SEQ ID NO: 4), or between K234 and E265 in mGSDMA3 (SEQ ID NO:2).

In other aspects, the photoactivable linker is inserted into the gasdermin amino acid sequence at the endogenous protease cleavage site, thereby disrupting recognition by the protease. In other aspects, the photoactivable linker is inserted at the endogenous protease cleavage site of human gasdermin A, human gasdermin B, human gasdermin C, human gasdermin D, human gasdermin E, human pejvakin, or a fragment or ortholog thereof. In particular aspects, the photocleavable linker insertion site is at the protease cleavage site QTFPPGE (SEQ ID NO:17) in human gasdermin A; DVLNSLA (SEQ ID NO:18) or DELDSGL (SEQ ID NO:19) in human gasdermin B; SSNDMKL (SEQ ID NO:20) in human gasdermin C; FLTDGVP (SEQ ID NO:21) in human gasdermin D; or DMPDAAH (SEQ ID NO:22) in human gasdermin E.

One of skill in the art can readily appreciate that a shift of several amino acids at the insertion point will still result in modified gasdermin domains that are functionally the same.

As used herein a photoactivatable linker is a protein component derived from fluorescent proteins or peptides, which, upon illumination, dimerize and/or photocleave to thereby control the pore dynamics of the modified gasdermin protein of this invention. Accordingly, in one aspect, the photoactivatable linker is an optogenetic dimerization protein. In another aspect, the photoactivatable linker is a photocleavable linker.

In one aspect, the photoactivatable linker is an optogenetic dimerization protein or light inducible dimerization protein. Optogenetic dimerization exploits a pair of specialized protein domains that can be driven into a high-affinity binding state by illumination with a specific wavelength of light. In accordance with this invention, a dimerization domain is genetically inserted into a gasdermin protein thereby allowing for experimental control over oligomeric pore formation. Exemplary optogenetic dimerization proteins of use in the modified gasdermin protein of this invention include, but are not limited to, VVD (Vivid), CRY (cryptochrome), CIBN (N-terminal domain of CIB1 (cryptochrome-interacting basic-helix-loop-helix protein 1)), PHY (phytochrome), PIF (phytochrome interacting factor), UVR8 (UV-B photoreceptor), FKF1 (Flavin-binding, Kelch repeat, F-box 1), GIGANTEA, TULIPS, Dronpa, iLID, AsLOV variant or combinations thereof.

VVD is a light-sensitive protein involved in the blue-regulated cell signaling pathway. Under blue light, it can react with flavin adenine dinucleotide (FAD, Flavin Adenine Dinucleotide) to form a dimer. The full-length VVD protein contains 186 amino acids and contains only one light-sensitive LOV domain. Studies have shown that the VVD protein lacking the N-terminal 36 amino acid residues (VVD36) is more stable than the full-length protein. In addition, VVD mutants Ile74Val and Ile85Val have been shown to facilitate dissociation of VVD dimers when placed in the dark (Zoltowski et al. (2009) Nat. Chem. Biol. 5:827-834). This enables faster reversibility of light-mediated changes. Mutations of Met135 and Met165 to Ile strengthen dimer binding (Zoltowski et al. (2009) Nat. Chem. Biol. 5:827-834). These mutants have been used previously to fine-tune light-mediated regulation of VVD dimerization (Zoltowski et al. (2009) Nat. Chem. Biol. 5:827-834; Kawano et al. (2015) Nat. Commun. 6:6256). Accordingly, one or more of these mutations may be introduced into the VVD sequence to modulate the kinetics of light activation. Representative VVD proteins of use in this invention are provided under UniProtKB Accession No. Q1K5Y8 and GENBANK Accession No. XP_957606.

“Cryptochrome” or “CRY” is an ultraviolet-A/blue light photoreceptor found in plants, insects, fish, amphibians, mammals and fungi. Cryptochromes are composed of two major domains, the N-terminal PHR (for Photolyase-Homologous Region) and the C-terminal extension CCE (for Cryptochrome C-terminal Extension) domain. The PHR domain is required for chromophore-binding and homodimerization (Sang et al. (2005) Plant Cell 17:1569-84; Yu et al. (2007) Proc. Natl. Acad. Sci. USA 104:7289-94), whereas CCE is an effector domain of cryptochrome (Yang et al. (2000) Cell 103: 815-827; Wang et al. (2001) Science 294:154-158). CRY proteins are known in the art and include those obtained from, e.g., Chlamydomonas reinhardtii, Physcomitrella patens (GENBANK Accession No. XP_001751763), Adiantum capillus-veneris, Arabidopsis thaliana (GENBANK Accession Nos. NP_567341 and NP_171935), Lycopersicon esculentum (GENBANK Accession No. NP_001234667), Sorghum bicolor (GENBANK Accession Nos. XP_002436988 and AAV97867), Oryza sativa (GENBANK Accession Nos. BAD17529 and BAD23780), Glycine max (GENBANK Accession Nos. NP_001242152 and NP_001235220) and Sinapis alba (Lin & Todo (2005) Genome Biology 6:220). A CRY of this invention may be composed of the PHR and CCE domains or only the PHR domain which has shown to be sufficient for light-dependent conformational changes (WO 2019/084362). While CRY-CRY homodimers are contemplated, a CRY-CIBN heterodimer is also included within the scope of this invention (see Liu et al. (2008) Science 322 (5907): 1535-9).

“CIBN” as used herein refers to the N-terminus of CIB that interacts with cryptochrome (CRY) upon irradiation with light. As used herein, “CIB” refers to cryptochrome-interacting basic-helix-loop-helix protein and is represented by the Arabidopsis CIB1 provided under GENBANK Accession No. NM 119618.

The phytochromes (PHY) include a family of biliprotein photoreceptors that enable plants to adapt to their prevailing light environment. PHY domains are excitable by red light, i.e., by light having a wavelength in the range of 600-690 nm, preferably 610-680 nm, more preferably in the range of 620-670 nm, and most preferably in the range of 630-660 nm, such as by light having a wavelength of about 650 nm. In addition, the light sensing PHY domain can be inactivated by light with a wavelength in the range of 700-750 nm, preferably 710-740 nm, more preferably 720-730 nm. Phytochromes from cyanobacteria to green algae and higher plants are composed of a well conserved N-terminal domain, roughly 390-600 amino acids in length (see, e.g., U.S. Pat. No. 6,046,014), to which the phytobilin prosthetic group is bound. An exemplary phytochrome sequence is disclosed in US 2003/0082809. Additional Phy proteins include Arabidopsis PhyA provided under GENBANK Accession No. NM 001123784 and PhyB provided under GENBANK Accession No. NM 127435. While PHY-PHY homodimers are contemplated, a PHY-PIF heterodimer is also included within the scope of this invention (see WO 2013/133643; Kim et al. (2014) Chem. Biol. 21:903-912).

As used herein, “PIF” refers to a phytochrome interacting factor, which is represented by the Arabidopsis PIF1, PIF3, PIF4, PIF5, PIF6, or PIF7 proteins respectively provided under Accession GENBANK Nos. NM 001202630, NM 179295, NM 180050, NM 180690, NM 001203231, and NM 125520.

“UVR8” is a seven-bladed β-propeller protein of 440 amino acid residues in length (Christie et al. (2012) Science 335: 1492-1496; Wu et al. (2012) Nature 484: 214-219). Molecular and biochemical studies have demonstrated that in light conditions devoid of UV-B, the UVR8 photoreceptor exists as a homodimer, which undergoes instant monomerization following UV-B exposure, a process dependent on an intrinsic tryptophan residue that serves as an UV-B chromophore (Rizzini et al. (2011) Science 332:103-106). Accordingly, in some embodiments, dimerization is induced in the absence of UV-B light. Alternatively, when used in combination with COP1, a light-induced UVR8-COP1 heterodimer can be formed (Rizzini et al. (2011) Science 332:103-106; Crefcoeur et al. (2013) Nat. Commun. 4:1779).

“FKF” refers to Flavin-binding, Kelch repeat, F-box proteins, typically FKF1 (GENBANK Accession No. NM_105475) of Arabidopsis.

Dronpa refers to a refers to photoreceptive polypeptide from a coral of the genus Pectiniidae. Dronpa rapidly converts between a dark state and a bright state upon illumination with 490 nm and 400 nm light, respectively. Therefore, Dronpa mutants that either dimerize in the bright state but remain monomeric in the dark state have been generated and fused to proteins such as a guanine nucleotide exchange factor (GEF) or protease (Zhou et al. (2013) Science 338 (6108):810-4). When in the bright state, the two Dronpa domains form an interface and upon exposure to 400 nm light, the interface breaks. Representative Dronpa are provided under GENBANK Accession Nos. AB180726, ADE48854, and BAD72874.1.

The tunable light-controlled interacting protein tags (TULIPs) make use of a blue light-sensing LOV domain and an engineered PDZ domain. Specifically, the LOV2 domain of Avena sativa phototropin 1 (AsLOV2) and an engineered PDZ domain (ePDZ) may be used. See Strickland et al. (2012) Nat. Methods 9(4): 379-384.

In alternative aspects, the photoactivatable linker is a photocleavable protein. As used herein, “photocleavable” or “photocleave” means the breaking of a covalent bond within the amino acid sequence of the protein upon illumination of the protein or peptide with light having a suitable wavelength and energy, thereby creating a new C-terminus and a new N-terminus. In a particular aspect, the photocleavable protein is a PhoCl protein. “PhoCl” refers to a photocleavable polypeptide comprising a His-Tyr-Gly chromophore, wherein the protein spontaneously dissociates into at least two fragments, or releases one end of a loop insertion, following photocleavage. PhoCl proteins are described in U.S. Pat. No. 10,370,420, incorporated herein by reference in its entirety. Exemplary PhoCl proteins include, e.g., PhoCle0.1, PhoCle0.2, PhoCle0.3, PhoCle0.4, PhoCle0.5, PhoCle0.6, PhoCle0.7, and cpPhoCle, as well as second generation PhoCl variants (PhoC12), e.g., the PhoC12c variant with higher dissociation contrast ratio and PhoC12f the variant with faster dissociation rate. Lu et al. (2021) Chem. Sci. 12(28): 9658.

In certain aspects, the PhoCl polypeptide comprises or consists of an amino acid sequence selected from SEQ ID NO:23, SEQ ID NO: 24, SEQ ID NO: 25, SEQ ID NO: 26, SEQ ID NO: 27, SEQ ID NO:28, SEQ ID NO:29, SEQ ID NO: 30, or SEQ ID NO:31, or a substantially similar amino acid sequence wherein the polypeptide is photocleavable and spontaneously dissociates into at least two fragments, or releases an end of an internal loop, upon photocleavage. The polypeptides described herein can be modified and varied so long as the desired function is maintained. In one embodiment, the invention provides a polypeptide comprising an amino acid sequence having at least 95%, at least 96%, at least 97%, at least 98%, or at least 99% sequence identity to one of SEQ ID NO: 23, SEQ ID NO: 24, SEQ ID NO:25, SEQ ID NO:26, SEQ ID NO:27, SEQ ID NO: 28, SEQ ID NO:29, SEQ ID NO: 30, or SEQ ID NO: 31, and wherein the encoded polypeptide is photocleavable and dissociates into at least two fragments, or releases an end of an internal loop. For example, the variant having substantial sequence identity may have no more than a 10% decrease or increase in function, and preferably no more than a 5% decrease or increase in function.

Those skilled in the art will appreciate that modifications (i.e., amino acid substitutions, additions, deletions and post-translational modifications) can be made to the photoactivatable linker without eliminating or diminishing its biological activity. Some modifications may be made to facilitate the cloning, expression or purification. Guidance in determining which amino acid residues can be substituted, inserted, or deleted without abolishing biological or immunological activity can be found using computer programs well known in the art, such as DNASTAR™ software. By way of illustration, the N-terminus and/or C-terminus may include flexible linker sequences, e.g., Gly-Gly-Gly or Gly-Gly-Gly-Ser (SEQ ID NO:32). Preferably, amino acid changes in the protein variants disclosed herein are conservative amino acid changes, i.e., substitutions of similarly charged or uncharged amino acids. Conservative amino acid substitutions (i.e., substitution of one amino acid for another amino acid of similar size, charge, polarity and conformation) or substitution of one amino acid for another within the same group (i.e., nonpolar group, polar group, positively charged group, negatively charged group) are unlikely to alter protein function adversely. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

In particular aspects, the modified gasdermin protein comprises or consists of the amino acid sequence set forth in SEQ ID NO: 33, SEQ ID NO: 34, SEQ ID NO: 35, SEQ ID NO: 36, SEQ ID NO:37, SEQ ID NO: 38, SEQ ID NO: 39 or SEQ ID NO: 40.

In another aspect, this invention provides an isolated nucleic acid molecule encoding the modified gasdermin protein of the invention. In a further aspect, the invention provides a recombinant vector harboring at least one isolated nucleic acid molecule encoding the modified gasdermin protein of the invention. In particular, the nucleic acid molecule encoding the modified gasdermin protein is inserted into a vector capable of delivering the nucleic acid molecule into a host cell. Such a vector contains heterologous nucleic acid sequences that are not naturally found adjacent to the nucleic acid molecule encoding the modified gasdermin protein and are preferably derived from a species other than the species from which the nucleic acid molecule encoding the modified gasdermin protein is derived. The vector can be either prokaryotic or eukaryotic, and typically is a virus or a plasmid. Recombinant vectors can be used in the cloning, sequencing, and/or otherwise manipulating the nucleic acid molecules of the present invention.

The present invention also includes an expression vector, which includes a nucleic acid molecule encoding the modified gasdermin protein of the invention in a recombinant vector that is capable of expressing the nucleic acid molecule when transformed into a host cell. Preferably, the expression vector is also capable of replicating within the host cell. Expression vectors can be either prokaryotic or eukaryotic and are typically viruses or plasmids. Expression vectors of the present invention include any vectors that function (i.e., direct gene expression) in recombinant cells such as bacterial, fungal, parasite, insect, other animal, and plant cells. Preferred expression vectors of the present invention can direct gene expression in bacterial, yeast, helminth or other parasite, insect and mammalian cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, helminth or other endoparasite, or insect and mammalian cells, such as, but not limited to, tac, lac, trp, trc, oxy-pro, omp/lpp, rrnB, bacteriophage lambda (such as lambda pr and lambda pR and fusions that include such promoters), bacteriophage T7, T7lac, bacteriophage T3, bacteriophage SP6, bacteriophage SP01, metallothionein, alpha-mating factor, Pichia alcohol oxidase, alphavirus subgenomic promoter, antibiotic resistance gene, baculovirus, Heliothis zea insect virus, vaccinia virus, herpesvirus, raccoon poxvirus, other poxvirus, adenovirus, cytomegalovirus (such as immediate early promoter), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with parasitic helminths, such as B. malayi transcription control sequences.

Recombinant molecules of the present invention may also contain (a) secretory signals (i.e., signal segment nucleic acid sequences) to enable an expressed protein of the present invention to be secreted from the cell that produces the protein and/or (b) fusion sequences which lead to the expression of nucleic acid molecules of the present invention as fusion proteins. Examples of suitable signal segments include any signal segment capable of directing the secretion of a protein of the present invention. Preferred signal segments include, but are not limited to, tissue plasminogen activator (t-PA), interferon, interleukin, growth hormone, histocompatibility and viral envelope glycoprotein signal segments. In addition, a nucleic acid molecule of the present invention can be joined to a fusion segment that directs the encoded protein to the proteosome, such as a ubiquitin fusion segment. Eukaryotic recombinant molecules may also include intervening and/or untranslated sequences surrounding and/or within the nucleic acid sequences of nucleic acid molecules of the present invention.

Another embodiment of the invention includes a recombinant mRNA vector which includes at least one mRNA molecule comprising at least one region coding for the modified gasdermin protein of the invention.

To increase the stability and transfection efficiency of the (m)RNA, each (m)RNA to be introduced into the host cells of the present invention preferably has one or more modifications, especially chemical modifications, which improve the transfer of the (m)RNA(s) into the cells to be transfected and/or increase the expression of the encoded antigen(s).

For example, the sequences of eukaryotic mRNAs contain destabilizing sequence elements (DSEs) to which signal proteins bind and regulate the enzymatic degradation of the mRNA in vivo. Therefore, for further stabilization of the mRNA, one or more changes are optionally made in the region coding for the modified gasdermin protein, relative to the corresponding region of the wild-type mRNA, so that no destabilizing sequence elements are present. Of course, it is also preferred according to the invention to eliminate from the mRNA any DSEs present in the untranslated regions (3′- and/or 5′-UTR).

Examples of such destabilizing sequences are AU-rich sequences (“AU-RES”), which occur in 3′-UTR segments of numerous unstable RNAs (Caput at al. (1986) Proc. Natl. Acad. Sci. USA 83:1670-1674). The RNA molecules used in the present invention are therefore preferably changed, relative to the wild-type mRNA, in such a way that they do not have any such destabilizing sequences. This also applies to sequence units (motifs) recognized by possible endonucleases. These sequence units (motifs) are also preferably eliminated from the modified mRNA used for transfection of the blood cells.

Those skilled in the art are familiar with various methods that are suitable for substituting codons in the mRNA modified according to the invention. In the case of shorter coding regions (coding for biologically effective or antigenic peptides), it is possible, for example, to synthesize the total mRNA chemically using standard techniques.

Preferably, however, base substitutions are introduced using a DNA template in order to prepare the modified mRNA by common directed mutagenesis techniques (Maniatis at al. (2001) Molecular Cloning: Laboratory Manual, Cold Spring Harbor Laboratory Press, 3^{rd} ed., Cold Spring Harbor, NY). In this method, therefore, an appropriate DNA molecule is transcribed in vitro in order to prepare the mRNA. This DNA template has a suitable promoter, e.g., a T7 or SP6 promoter, for the in vitro transcription, which is followed by the desired nucleotide sequence for the mRNA to be prepared and by a termination signal for the in vitro transcription. According to the invention, the DNA molecule that forms the template of the RNA construct to be prepared is conventionally prepared by fermentative multiplication and subsequent isolation as part of a plasmid replicable in bacteria. Examples which may be mentioned of plasmids suitable for the present invention are pT7TS (GENBANK Accession Number U26404; Lai et al. (1995) Development 121:2349-2360), the pGEM® series, e.g., pGEM®-1 (GENBANK Accession Number X65300; from Promega), and pSP64 (GENBANK Accession Number X65327) (cf. also Mezei & Storts (2001) Purification of PCR Products, in Griffin and Griffin (eds), PCR Technology: Current Innovation, CRC Press, Boca Raton, FL).

Thus, the desired nucleotide sequence can be cloned into a suitable plasmid by molecular biological methods known to those skilled in the art using short synthetic DNA oligonucleotides which have short single-stranded transitions at the existing restriction sites, or using genes prepared by chemical synthesis. The DNA molecule is then cleaved from the plasmid, in which it can be present in single or multiple copy, by digestion with restriction endonucleases.

The modified mRNA which can be used for transfection of the cells can also have a 5′ cap structure (a modified guanosine nucleotide). Examples of cap structures which may be mentioned are m7G(5′)ppp, (5′)A,G(5′)ppp(5′)A and G(5′)ppp(5′)G.

In another aspect of the present invention, the modified mRNA contains a poly(A⁺) tail of at least about 25, especially of at least about 30, preferably of at least about 50, particularly preferably of at least about 70 and very particularly preferably of at least about 100 nucleotides. However, the poly(A⁺) tail can also comprise 200 nucleotides or more.

Efficient translation of the mRNA further requires an effective binding of the ribosomes to the ribosome binding site (i.e., Kozak sequence). It has been found in this regard that an increased A/U content around this site enables a more efficient ribosome binding to the mRNA.

It is also possible to insert one or more so-called IRESs (internal ribosomal entry sites) into the mRNA. An IRES can thus act as a single ribosome binding site but it can also be used to provide an mRNA coding for several (e.g., two) peptides or polypeptides which are to be translated independently of one another by the ribosomes in the PEMCs (“multicistronic” or “polycistronic” (e.g., bicistronic) mRNA). Examples of IRES sequences that can be used according to the invention are those from picornaviruses (e.g., FMDV), plague viruses (CFPV), polioviruses (PV), encephalomyocarditis viruses (ECMV), foot-and-mouth disease viruses (FMDV), hepatitis C viruses (HCV), classical swine fever viruses (CSFV), murine leukemia virus (MLV), simian immunodeficiency viruses (SIV) or cricket paralysis viruses (CrPV).

In another aspect of the present invention, in the 5′- and/or 3′-untranslated regions, the mRNA has stabilizing sequences capable of increasing the half-life of the mRNA in the cytosol. These stabilizing sequences can have a 100% sequence homology to naturally occurring sequences that appear in viruses, bacteria and eukaryotes, but they can also be of a partially or completely synthetic nature. Examples which may be mentioned of stabilizing sequences that can be used in the present invention are the untranslated sequences (UTR) of the α and β-globin gene, e.g., from Homo sapiens or Xenopus laevis. Another example of a stabilizing sequence is that present in the 3′-UTR of the very stable mRNA coding for α-globin, α-(1)-collagen, 15-lipoxygenase or tyrosine hydroxylase (Holcik et al. (1997) Proc. Natl. Acad. Sci. USA 94:2410-2414). Of course, such stabilizing sequences can be used individually, in combination with one another or in combination with, other stabilizing sequences known to those skilled in the art.

For further stabilization, the mRNA may also have at least one analogue of naturally occurring nucleotides. This is based on the fact that the RNA-degrading enzymes occurring in the blood cells preferentially recognize naturally occurring nucleotides as substrate. The RNA degradation can therefore be made more difficult by inserting nucleotide analogues, it being possible for the insertion of these analogues, especially into the coding region of the mRNA, to have a positive or negative effect on the translation efficiency.

The following may be mentioned as examples of nucleotide analogues that can be used according to the invention, without the list in any way being definitive phosphoramidates, phosphorothioates, peptide nucleotides, methylphosphonates, 7-deazaguanosine, 5-methylcytosine and inosine. The preparation of such analogues is known to those skilled in the art, e.g., in U.S. Pat. Nos. 4,373,071; 4,401,796, 4,415,732; 4,458,066; 4,500,707; 4,668,777; 4,973,679; 5,047,524; 5,132,418; 5,153,319; 5,262,530 and 5,700,642. According to the invention, such analogues can occur in untranslated and translated regions of the modified mRNA.

Furthermore, the effective transfer of the preferably modified mRNA into the cells can be improved if the mRNA is associated with or bound to a cationic or polycationic agent, especially an appropriate peptide or protein, prior to transfection of the previously obtained blood cells. The mRNA is therefore preferably complexed or condensed with such an agent prior to transfection of the PBMCs. It is particularly effective here to use protamine as a polycationic, nucleic acid-binding protein. It is also possible to use other cationic peptides or proteins, such as poly-L-lysine, poly-L-arginine or histones. This procedure for stabilizing the modified mRNA is described in EP 1083232. The mRNA for transfection into the cells can also be associated or mixed with other substances for efficient transfer. Examples of this are inclusion in microparticles or nanoparticles, especially those based on PLGA (poly(D,L-lactide-co-glycolide)), and lipids.

Furthermore, according to the invention, the mRNA can also contain, in addition to the modified gasdermin protein encoding sequence, at least one other functional segment coding, e.g., for a cytokine that promotes the immune response (monokine, lymphokine, interleukin or chemokine, such as IL-1, IL-2, IL-3, IL-4, IL-5, IL-6, IL-7, IL-8, Its-9, IL-10, IL-12, IFN-α, IFN-γ, GM-CSF and LT-α) or growth factors such as hGH. Alternatively or additionally, the mRNA provided for transfection of the blood cells or haemopoietic cells, especially red blood cells, PBMCs, granulocytes and/or blood platelets, can also code for at least one co-stimulating molecule (e.g., CD40, CD80, CD86 or 4-1BB ligand) and/or at least one transcription factor (e.g., NF-kappaB or ICSBP (interferon consensus binding protein)), which assures a particularly efficient expression of immunostimulating molecules in the transfected cells, and/or for at least one homing receptor (e.g., CCR7), which directs the transfected cells, e.g., into the lymph nodes, and/or for at least one suicide molecule (e.g., herpes simplex virus thymidine kinase (HSV-tk), cytochrome P450 4B1 (cyp4B1) and/or folylpolyglutamate synthase (FPGS)), which is expressed in the transfected cells and converts an otherwise inactive prodrug to its active form (e.g., nucleoside analogues, such as ganciclovir or acyclovir, by HSV-tk, and/or 4-ipomeanol or 2-aminoanthracene by cyp4B1), or intensifies the action of a chemotherapeutic agent that is already effective per se alkylating agents, such as methotrexate, by FPGS), and thereby induces necrotic and/or apoptotic cell death, resulting in the release of the cell contents, which include the antigen encoded by the mRNA.

Another aspect of the present invention includes a recombinant host cell harboring one or more recombinant molecules of the present invention. Transformation of a nucleic acid molecule or vector into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules or vectors of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a nucleic acid molecule or vector of the present invention. Host cells can be either untransformed cells or cells that are already transformed with at least one nucleic acid molecule (e.g., nucleic acid molecules encoding modified gasdermin protein and/or other proteins described herein). Host cells of the present invention include bacterial, fungal (including yeast), parasite (including helminth, protozoa and ectoparasite), other insect, other animal and plant cells. Preferred host cells include bacterial, mycobacterial, yeast, helminth, insect and mammalian cells. More preferred host cells include Salmonella, Escherichia, Bacillus, Listeria, Saccharomyces, Spodoptera, Mycobacteria, Trichoplusia, BHK (baby hamster kidney) cells, MDCK cells (Madin-Darby canine kidney cell line), CRFK cells (Crandell feline kidney cell line), CV-1 cells (African monkey kidney cell line used, for example, to culture raccoon poxvirus), COS (e.g., COS-7) cells, and Vero cells. Particularly preferred host cells are Escherichia coli, including E. coli K-12 derivatives; Salmonella typhi; Salmonella typhimurium; Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK31 cells, Hela cells and/or RAW264.7 cells.

A recombinant cell preferably produced by transforming a host cell with one or more recombinant molecules, each comprising one or more nucleic acid molecules of the present invention and one or more transcription control sequences, examples of which are disclosed herein.

Alternatively, a modified gasdermin protein can be produced using CRISPR techniques to introduce the photoactivable linker between the N-terminal domain and C-terminal domain of a gasdermin protein encoded by the genome of a host cell.

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein. Moreover, while non-codon-optimized sequences may be used to express fusion proteins in host cells such as E. coli, the nucleic acid molecule may be codon-optimized to facilitate expression in mammalian cells.

Modified gasdermin proteins of the invention can be produced in a variety of ways, including production and recovery of recombinant proteins or chemical synthesis of the proteins. In one aspect, an isolated protein of the present invention is produced by culturing a cell capable of expressing the protein under conditions effective to produce the protein, and recovering the protein. A preferred cell to culture is a recombinant cell of the present invention. Effective culture conditions include, but are not limited to, effective media, bioreactor, temperature, pH and oxygen conditions that permit protein production. An effective, medium refers to any medium in which a cell is cultured to produce a protein of the present invention. Such medium typically includes an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

Recovery of proteins of invention can include collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins of the present invention are preferably retrieved in substantially pure form thereby allowing for the effective use of the protein, e.g., as a therapeutic composition. A therapeutic composition for animals, for example, should exhibit no substantial toxicity and preferably should be capable of stimulating the production of antibodies in a treated animal.

Compositions containing the modified gasdermin proteins, nucleic acids, vectors, and cells of the invention can be prepared by combining the modified gasdermin proteins, nucleic acids, vectors, and cells with a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and the like. The use of such media and agents for pharmaceutically active substances is well known in the art. Except insofar as any conventional media or agent is incompatible with the modified gasdermin proteins, nucleic acids, vectors, and cells of the present disclosure, its use in therapeutic compositions is contemplated. Pharmaceutical compositions can be determined by one skilled in the art depending upon, for example, the intended route of administration, delivery format and desired dosage. See, for example, Remington's Pharmaceutical Sciences, 18th Edition (1990) A. R. Gennaro, ed., Mack Publishing Company.

The pharmaceutical composition of the invention can be incorporated in an injectable formulation. The formulation may also include the necessary physiologically acceptable carrier material, excipient, lubricant, buffer, surfactant, antibacterial, bulking agent (such as mannitol), antioxidants (ascorbic acid or sodium bisulfite) and the like.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials may include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA; complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as PEG, sorbitan esters, polysorbates such as polysorbate 20 and polysorbate 80, TRITON, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington's Pharmaceutical Sciences, Id.

The primary vehicle or carrier in a pharmaceutical composition may be either aqueous or nonaqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, which may further include sorbitol or a suitable substitute therefore. Pharmaceutical compositions of the invention may be prepared for storage by mixing the selected composition having the desired degree of purity with optional formulation agents (Remington's Pharmaceutical Sciences, Id.) in the form of a lyophilized cake or an aqueous solution.

The compositions can be provided by sustained release systems, by encapsulation or by implantation devices. The compositions may be administered by bolus injection or continuously by infusion, or by implantation device. The compositions also can be administered locally via implantation of a membrane, sponge or another appropriate material onto which the cell or cells have been absorbed or encapsulated. Where an implantation device is used, the device may be implanted into any suitable tissue or organ. The injections may be given as a one-time treatment, repeated (daily, weekly, monthly, annually etc.) in order to achieve the desired therapeutic effect.

The pharmaceutical composition of the invention can be delivered parenterally. When parenteral administration is contemplated, the pharmaceutical composition for use in this invention may be in the form of a pyrogen-free, parenterally acceptable aqueous solution. A particularly suitable vehicle for parenteral injection is sterile distilled water. Preparation can involve the formulation with an agent, such as injectable microspheres, bio-erodible particles, polymeric compounds (such as polylactic acid or polyglycolic acid), beads or liposomes, that may provide controlled or sustained release of the cell or cells, which may then be delivered via a depot injection. Formulation with hyaluronic acid has the effect of promoting sustained duration in the circulation. Implantable drug delivery devices may be used to introduce the desired composition.

These compositions may also contain adjuvants such as preservative, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like.

Supplementary active ingredients also can be incorporated into the compositions. The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intraperitoneal, or intravenous injection. Such compositions would normally be administered as pharmaceutically acceptable compositions.

In addition to compositions, this invention also provides methods of controlling or modulating gasdermin pore formation, in particular pore formation dynamics and pore size. In accordance with these methods, a photoactivatable linker, in particular a photocleavable linker, is inserted between the N-terminal domain and C-terminal domain of a gasdermin protein of a cell or subject and the cell or subject is illuminated with light having a wavelength suitable to activate the photoactivatable linker so that pore formation, e.g., pore size, is modulated. In particular aspects, said illumination with light will increase pore size. Using these methods of the invention, pore formation is programmable in that timing, size and subcellular localization of gasdermin pore formation can be precisely modulated with or without cell death, depending on length of illumination. These methods find use in a research setting to monitor cellular activities in response to efflux and/or influx of molecules via the gasdermin pore and for carrying out cell content biopsy, as well as a therapeutic setting for treating a disease or condition in a subject wherein gasdermin pore formation in a cell of the subject confers a benefit, e.g., a subject with faulty homeostasis of reactive oxygen species (ROS). See, e.g., U.S. Pat. No. 10,751,385 B2, incorporated herein by reference in its entirety, which describes the use of gasdermin for modulating cellular redox homeostasis in a suffering from an age-related disease selected from Parkinson's disease (PD), Alzheimer's disease (AD), Familial Amyotrophic Lateral Sclerosis (FALS), age-related macular degeneration (ARMD), type 2 diabetes, atherosclerosis, arthritis, cataracts, osteoporosis, hypertension, skin aging, skin pigmentation, and cardiovascular diseases; a patient suffering from a peroxisomal disorder leading to ROS production, or a patient suffering from a mitochondrial disorder leading to ROS production. In addition, having demonstrated that PI3K/PLC enzymes form a major signaling circuit in regulating oligomeric Gasdermin D pore activity, cytokine release and hence the strength of the downstream inflammatory signals can be modulated by a combination of light and PI3K/PLC modulators. In addition, optogenetic activation of gasdermin via the present modified gasdermin protein of this invention can kill cells via pyroptosis to an extent that is the same or better than known activation of natural pyroptosis cell death. Accordingly, increasing gasdermin pore formation dynamics and effective pore size is of use in cell killing, in particular cancer cell killing. Such cell killing can be used in conjunction with transplanted cells to reprogram their functions, thereby having an impact, for example, on the cancer killing ability of CAR-T cells. The methods and compositions may also find use in diseases where cytokine storm is pathological and immune cells require reprogramming. In vivo illumination schemes may also have also been used in surgical or hemodialysis settings.

In a further aspect, this invention provides methods for facilitating transport of a therapeutic agent into a cell and facilitating treatment of a disease or condition in a subject with a therapeutic agent. In accordance with these methods, a photoactivatable linker, in particular a photocleavable linker, is inserted or introduced between the N-terminal domain and C-terminal domain of a gasdermin protein of a cell or subject; a therapeutic agent is provided to the cell or subject; and the cell or subject is illuminated with or exposed to light having a wavelength suitable to activate the photoactivatable linker and modulate gasdermin pore formation, e.g., size, timing of opening, and/or location, thereby facilitating transport of the therapeutic agent into the cell or facilitating treatment of the disease or condition in the subject with the therapeutic agent. In particular aspects, said illumination with or exposure to light will increase pore size. Using these methods of the invention, pore formation is programmable in that timing, size and subcellular/cellular localization of gasdermin pore formation can be precisely modulated without cell death. These methods find use in a research setting to monitor cellular activities in response to the administration of a therapeutic agent, as well as a therapeutic setting for treating a disease or condition in a subject wherein gasdermin pore formation can be used to selectively increase transport of the therapeutic agent into one or more particular cell types. Notably, use of a modified gasdermin protein in conjunction with a therapeutic agent will permit targeted delivery of the therapeutic agent to a cell, tissue or organ by illumination of the cell, tissue or organ with an appropriate wavelength to activate the photoactivatable linker. In this respect, the instant methods find application in super resolution surgery, drug/gene delivery, and repeated “sutures” over time.

As used herein, an “active agent” or “therapeutic agent” refers to any substance that, when administered in a therapeutically effective amount to a patient suffering from a condition, has a therapeutic beneficial effect on the health and well-being of the patient. A therapeutic beneficial effect on the health and well-being of a patient includes, but it not limited to: (1) curing the condition; (2) slowing the progress of the condition; (3) causing the condition to retrogress; or, (4) alleviating one or more symptoms of the condition. As used herein, an active agent also includes any substance that when administered to a patient, known or suspected of being particularly susceptible to a condition, in a prophylactically effective amount, has a prophylactic beneficial effect on the health and well-being of the patient. A prophylactic beneficial effect on the health and well-being of a patient includes, but is not limited to: (1) preventing or delaying on-set of the condition in the first place; (2) maintaining a condition at a retrogressed level once such level has been achieved by a therapeutically effective amount of a substance, which may be the same as or different from the substance used in a prophylactically effective amount; or, (3) preventing or delaying recurrence of the condition after a course of treatment with a therapeutically effective amount of a substance, which may be the same as or different from the substance used in a prophylactically effective amount, has concluded. An “active agent” herein may include any therapeutic agent that is known or suspected to have a beneficial affect with regard to a cell-borne disease or disorder. Representative active agents include DNA, RNA, small organic molecules, and affinity reagents including antibodies, nanobodies, and the like, as well as therapeutic agents that are known to be cell impermeant, i.e., therapeutic agents that cannot cross the cell membrane efficiently.

As used herein, the term “amount effective,” “effective amount” or a “therapeutically effective amount” refers to an amount of the modified gasdermin proteins, nucleic acids, vectors, and cells or composition of the invention sufficient to achieve the desired result. The amount of the cells or composition which constitutes an “effective amount” or “therapeutically effective amount” may vary depending on the severity of the disease, the condition, weight, or age of the patient to be treated, the frequency of dosing, or the route of administration, but can be determined routinely by one of ordinary skill in the art. A clinician may titer the dosage or route of administration to obtain the optimal therapeutic effect.

Although not precluded, treating a disease or condition does not require that the disease, condition, or symptoms associated therewith be completely eliminated, including the treatment of acute or chronic signs, symptoms and/or malfunctions. “Treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. “Treatment” therefore also includes relapse prophylaxis or phase prophylaxis. The term “treat” and synonyms contemplate administering a therapeutically effective amount of the modified gasdermin proteins, nucleic acids, vectors, and cells or composition of the invention to an individual in need of such treatment. A treatment can be oriented symptomatically, for example, to suppress symptoms. Treatment can be carried out over a short period, be oriented over a medium term, or can be a long-term treatment, for example within the context of a maintenance therapy.

The wavelength of light used in the methods of this invention to activate the photoactivatable linker will be dependent upon the photoactivatable linker, i.e., the optogenetic dimerization protein or a photocleavable linker inserted into the modified gasdermin protein. When the modified gasdermin protein is used in the treatment of a disease or condition, the photoactivatable linker may be selected based upon the tissue to be targeted. For example, when the tissue being treated is muscles or nerves, a linker activatable with infrared light, photoactivatable e.g., wavelengths in the range of 700 nm to 1200 nm may be used. For tissue just beneath the skin, a photoactivatable linker activatable with red light, e.g., wavelengths in the range of 630 nm to 700 nm, or blue light, e.g., wavelengths in the range of 446 nm to 477 nm, may be used. For treatment of the skin, UV light may be used, e.g., UVA light in the range of 315 nm to 399 nm, UVB light in the range of 280 nm to 314 nm, or UVC light in the range of 100 nm to 279 nm. In addition, light intensity, duration and frequency can be modulated to precisely control pore formation with a modified gasdermin protein of this invention.

The following non-limiting examples are provided to further illustrate the present invention.

### Example 1: Materials and Methods

Protein Purification. Plasmid encoding the full length human GSDMD with a N-terminal His-SUMO tag was transformed into E. coli BL21 strain. The cultures were grown at 37° C. until OD₆₀₀ 0.8, and protein expression was induced with 0.2 mM isopropyl β-D-1-thiogalactopyranoside (IPTG) overnight at 20° C. The cells were harvested by centrifugation and the pellet was resuspended in 20 mM Tris buffer, pH 7.5, 50 mM NaCl, 5 mM imidazole, 20 mM MgCl₂, 10 mM KCl, 0.5 mM TCEP, 0.1 mM protease inhibitor, and DNase I. After resuspension, cells were disrupted by high-pressure and centrifuged at 30,000 g at 4° C. for 1 hour. The supernatant was incubated for 2 hours at room temperature with pre-equilibrated Ni-NTA affinity resin (Thermo Scientific) and then passed through a column for gravity flow purification. The column was washed with 20 column volumes of resuspension buffer, and the fusion protein was eluted with three column volumes of the same buffer with 250 mM imidazole. SUMO-tag cleavage was achieved by addition of ULP1 protease to the solution and subsequent dialysis overnight at 4° C. against 20 mM Tris buffer, pH 7.5, 50 mM NaCl, 0.5 mM TCEP. GSDMD was eluted from a second round of purification through pre-equilibrated Ni-NTA affinity resin. The protein was further purified by HI-TRAP® Q ion-exchange resin and a SUPERDEX® 75 gel filtration column (GE Healthcare) pre-equilibrated with 20 mM Tris buffer, pH 7.5, 50 mM NaCl, 0.5 mM TCEP. The purified protein was concentrated to 20 mg/ml and frozen at −80° C.

Free-Standing Lipid Bilayer Formation and Electrophysiology. For the lipid bilayer, a bilayer chamber with a polystyrene cuvette was used. In addition, the folding method, which is based on Langmuir-Blodgett film formation (Kroemer et al. (2009) Cell Death Differ. 16:3-11; Weavers et al. (2016) Cell 165:1658-1671) was employed. Monolayers that were spread in the air-water interface were faced at the chamber hole (Bergsbaken & Fink (2009) Nat. Rev. Microbiol. 7:99-109; Mulvihill et al. (2018) EMBO J. 37 (14): e98321). In brief, a shallow trough made of polystyrene was filled with recording solution, and the surface was separated into two parts by one of the cuvette walls. Bilayers were formed by monolayers of 1:3 1-palmitoyl-2-oleoylphosphatidylethanolamine (POPE)/1-palmitoyl-2-oleoylphosphatidylcholine (POPC) (Avanti-lipids) in pentane; where appropriate, phosphoinositides of the indicated headgroup were introduced with di-oleoyl (18:1) fatty acid chains at 10% relative concentration. A small amount of phospholipid, dispersed in pentane solution, was dropped on one of the water surfaces and as pentane evaporated, the lipid molecules remained at the air-water interface. The movements of the lipid molecules became less vigorous, and eventually the lipid molecules packed together to form a monolayer. For the formation of the planar lipid bilayer by the folding method, a monolayer was formed at the air-water interface in each compartment. Upon raising the recording solution level by adding more solution, the monolayer ran up on the cuvette surface and passed through the hole, where the two monolayers from both sides encountered each other and formed a bilayer. The membrane formed by this method has been referred to as a “solvent-free” membrane. The advantage of this method over other methods, like the painting method, is the capacity to form an asymmetrical membrane in which the lipid composition of either leaflet is arbitrarily defined (Kayagaki et al. (2015) Nature 526: 666-671). Once formed, the membrane retains this asymmetry for a long time because the flip-flop or exchange of the lipid molecules between both leaflets of the membrane is very slow.

For incorporation of the GSDMD into the lipid bilayer, 2 μM of the purified GSDMD protein was added to the cis side chamber along with 2 μM of caspase 1. The specific membrane capacitance of the membrane formed by the folding method was 0.6-0.8 μF/cm². This value was close to the native biological membrane, indicating that practically no solvent layer existed between the two monolayers. After the formation of a stable lipid bilayer and after the addition of GSDMD and caspase 1 mix by perfusion system, the lipid bilayer was clamped at different voltages from 100 mV to −100 mV via a gap-free protocol using an Axopatch 200B amplifier (Molecular Devices) with a Digidata 1440A (Molecular Devices) to record while applying constant voltages to the lipid bilayer. The cis solution was composed of 135 mM CsSO₃CH₃, 8 mM NaCl, 2 mM MgCl₂, 0.5 mM CaCl₂, 2 mM EGTA and 10 mM HEPES at pH 7.2; and the trans solution was composed of 145 mM NaCl₂, 2 mM CaCl₂), 1 mM MgCl₂, 10 mM HEPES at pH 7.4. The data were analyzed using Clampfit and R2. The recording pipette was filled with internal solution. For controls, GSDMD or caspase 1 were each added alone. For the phospholipid incorporation, the same lipid bilayer formation technique was used, but the different phospholipids were added to the POPE/POPC mix at the cis side of the chamber before the bilayer was formed.

Cloning. Cloning and subcloning were performed using the DH5a strain of E. coli. All mammalian constructs were cloned into the pcDNA3.0 vector with a modified multiple cloning site. Plasmids were generated as follows. Gasdermin D gene was obtained from Addgene. A diffusible optogenetic GSDMD, referred to herein as “PhoDer” and set forth in SEQ ID NO: 33, was constructed according to the reported domains structure by ligating three fragments into the pcDNA3.0 vector in one reaction via Gibson Assembly (New England BioLabs). For each fragment, PCR was specifically designed to create appropriate complementation. The jRCaMP1b gene, encoding a sensitive red protein calcium indicator, was obtained from Addgene. Membrane targeted calcium indicator jRCaMP1b was constructed by ligating a PCR fragment of the calcium indicator into the restriction enzyme sites BamHI and EcoRI in a modified pcDNA3.0 vector carrying a 5′ fragment encoding the N-terminal Lyn kinase localization sequence.

Cell Culture. HeLa and RAW264.7 cells were maintained in Dulbecco's Modified Eagle Medium (DMEM) medium supplemented with 10% fetal bovine serum (FBS) and 1% penicillin and streptomycin. Cells were transfected at 50%-70% confluency using LIPOFECTAMINE® 2000 transfection reagent and incubated for 24 hours before imaging. Cells were never serum-starved. To examine endogenous inflammatory activation, RAW264.7 cells were treated with 100 ng/μL lipopolysaccharide (LPS, Invivogen) at the beginning of each experiment. Pretreatment to bias membrane composition by phosphatidylinositol 4,5-bisphosphate (PIP2) modifying enzymes was performed by incubating Hela cells in Hanks' Balanced Salt Solution (HBSS) buffer with inhibitors at 37° C. for 10 minutes to prior imaging at the following concentrations: for phosphoinositide 3-kinase (PI3K), cells were treated 10 μM wortmannin (Tocris); and for phospholipase C (PLC), cells were treated with 10 μM U73122 (Tocris). Cells were removed from the incubator and allowed to come to thermal equilibrium before being imaged in HBSS buffer at room temperature.

Fluorescence Imaging, Optogenetic Activation, and Post-Processing. All epifluorescence imaging was performed on a customized Nikon Ti2-E microscope equipped with individual control over excitation (EX) and emission (EM) filters, a fast LED light source (Lumencor) with fluid-cooled EMCCD (Andor), motorized stage, and examined under a 40× or 100× oil immersion objective under live focus tracking. For RFP, excitation (EX) and emission (EM) filter combinations (maxima/bandwidth in nm) are indicated. Cells were imaged at 12-15 second intervals. Emission intensities of individual cells were background-subtracted before normalization to zero time point, when optogenetic activation began. In the optical setup, optogenetic activation of PhoDer was performed using an EX filter at 440/20 nm for 20-30 ms across whole fields of view. This replicated the progression of endogenous activation of GSDMD observed in LPS-stimulated RAW264.7 cells. PhoDer activation through 395/25 nm EX filter led to a fast activation of GSDMD, causing rapid cell “bubbling,” blebbing, and death in calcium response time course comparable to nigericin treatment. Where applicable, inhibitors were treated at the indicated concentration for 15 minutes prior to imaging.

Fluctuation Analysis and Quantification. To automate the objective isolation and quantitation of calcium flares, a post-processing algorithm was developed based on principles and normalization scheme detailed in work on photochromic stochastic optical fluctuation imaging (pcSOFI) (Dedecker et al. (2012) Proc. Natl. Acad. Sci. USA 109: 10909-10914). Briefly, fluctuation caused by the flares generate high autocorrelation signals in time-averaged series and yield high contrast at locales of the greatest ion flux. To truly reflect the local flux change, it is then normalized to remove dependence on the number of biosensors reporting at the given location using a scheme detailed previously (Mo et al. (2017) Nat. Methods 14 (4): 427-434). Using Matlab, rolling-window mean and rolling-window autocorrelation values were calculated for each pixel over each entire fluorescence time course that contained possible flares. A heuristic approach to window size was taken and a range of window sizes were examined using examples where calcium flares were visually distinct prior to setting the window size for all analyses. All such fluctuation images were then verified manually against intensity time course to ensure bone fide flare detection; particularly strong and non-overlapping flares were chosen and added to calculate kinetic parameters. Furthermore, the high contrast capability of this method allowed flares to be reliably isolated via ImageJ threshold and particle analysis. The workflow was automated and applied to all PhoDer datasets and subsequently summarized.

Cytokine Release. LPS-stimulated, IL-1β cytokine release from bone-marrow monocyte-derived macrophages (C57b16 mice) was measured using the QUANTIKINE® ELISA kit (R&D Systems). Cells were seeded in 96-well plates and pre-treated for 10 minutes with medium, 10 μM U73122, or 10 μM wortmannin. LPS (100 ng/μL, Invivogen) was then added to the cells for 5 minutes and the cell-free supernatants were recovered and subjected to the ELISA kit according to the manufacturer's instructions. The concentration of the samples in 96-well plates was measured via absorbance at 450 nm using an Epoch Microplate Spectrophotometer (Biotek Instruments). The OD values in six technical repeats each from a total of three mice (n=18) were converted into the IL-18 levels reported via a power law fit of a serial dilution calibration curve.

Statistical Methods. The reported statistical significance between control and experimental datasets were the results of one-way ANOVA or two tailed, unpaired Welch's t-tests calculated at 95% confidence level using Graphpad Prism.

### Example 2: Optogenetic Activation of GSDMD Forms Pores and Recapitulates the Phenotype of Activated Macrophages and Fibroblasts

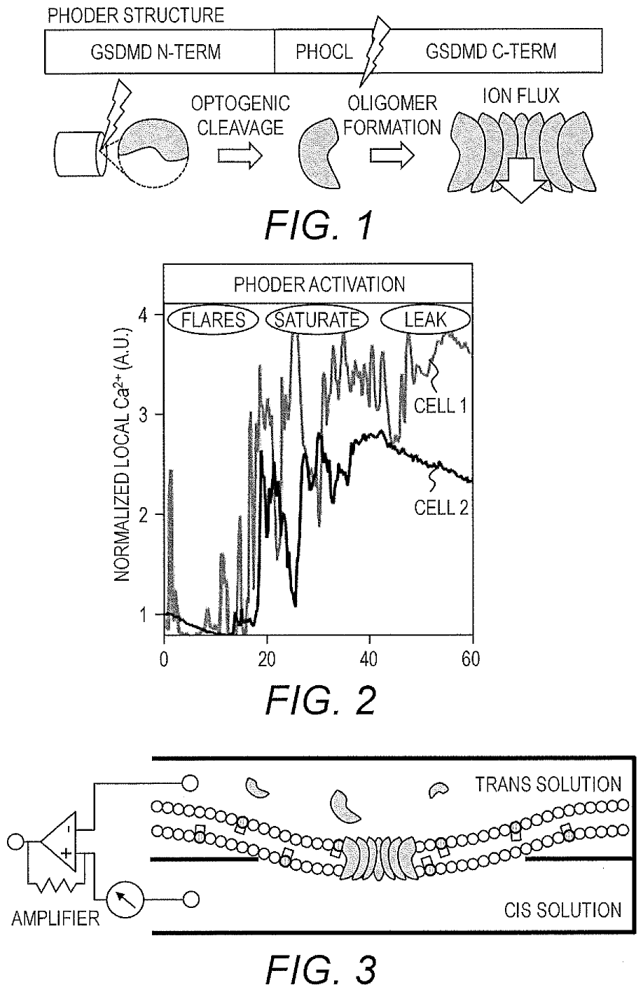

To study the dynamics of gasdermin pores, an optogenetically activatable human GSDMD was developed in which the C-terminal autoinhibitory domain is cleaved and released upon blue light illumination (FIG. 1), thereby allowing for precise and orthogonal analysis of GSDMD pore dynamics. As GSDMD pores mediate ion influx and efflux, it was examined whether the hypothesized pore dynamics would be reflected by transient calcium responses. In transiently transfected RAW264.7 and mouse bone marrow-derived macrophages (BMDM), activation of the diffusible optogenetic GSDMD (termed PhoDer) initiated clearly localized transient calcium flares. In Hela cells expressing PhoDer, optogenetic activation similarly caused spontaneous calcium flares at sporadic locations on the plasma membrane, which were visible via membrane-targeted jRCaMP1b29. Upon simple tuning, overt activation was avoided, cells did not lyse for tens of minutes, and calcium flare events were observed, followed by a gradual increase in whole-cell calcium, and finally calcium efflux and cell lysis as reported for pyroptosis (FIG. 2). Both flares and whole-cell calcium increases were absent when extracellular calcium was withheld, indicating that intracellular calcium stores did not contribute to these events. All cells (n=81 biologically independent cells in n>5 independent experiments) showed localized calcium flares and widespread whole-cell calcium fluctuations (FIG. 2); the large standard deviation indicated that the progression was asynchronous across the cell population. With further tuning, rapid PhoDer activation reproduced the severe blebbing “bubble” formation observed after nigericin-induced NLRP3 inflammasome activation in primed BMDMs. Controls lacking PhoDer expression lacked such calcium response. The design of PhoDer involved no other mechanism except the simple release of monomeric GSDMD autoinhibition, indicating that GSDMD alone was responsible for the calcium flares in macrophages and fibroblasts.

To further address whether the calcium dynamics primarily reflected that of GSDMD pores, whole-cell patching was directly recorded in living endothelial cells, which also undergo pyroptosis. Only the injection of activated, recombinant GSDMD proteins into these cells showed single pore currents that again marked repeated fast pore openings, stable current, and quick pore closures, whereas controls showed little response (n=15 for each). Additionally, as befitting a large pore, the GSDMD pore opening/closing dynamics were not restricted to calcium observations. Calcium flares were visualized, which occurred in coincidence with the transient flux of a large fluorogenic molecule, within the same spatiotemporal context in living cells. Furthermore, expression of membrane-targeted GSDMD-C(C-terminal domain of GSDMD) significantly dampened the optogenetically-induced calcium response, indicating that post-activation, GSDMD did not bind other partners and remained accessible to cognate inhibition. Therefore, observations across multiple platforms and perspectives show that GSDMD pores are highly dynamic in the live-cell plasma membranes.

### Example 3: Gasdermin D Pores Close Intact without Membrane Disruption

GSDMD oligomers form large pores without obstructions, unlike ion channels. The above results demonstrated that pore kinetics and size are not conducive to live-cell super-resolution imaging, making it difficult to address the closure of such supra-structures. However, the calcium influx does create an expanding wavefront while the pore remained open. Thus, it was surmised that the perimeter of this wavefront could be resolved to approximate the underlying, dynamic pore geometry. Utilizing PhoDer in combination with an algorithm similar to photochromic Stochastic Optical Fluctuation Imaging (pcSOFI; Dedecker et al. (2012) Proc. Natl. Acad. Sci. USA 109:10909-10914; Mo et al. (2017) Nat. Methods 14:427-434), the location of high calcium-change was highlighted to visualize each single pore during its open/close actions. The images were sufficiently sensitive to discern the geometry of pores above the slow-evolving intracellular calcium signals. Images were routinely generated that, while diffraction-limited, emphasized flares at a signal-to-background ratio exceeding three orders of magnitude. This enabled algorithmic detection and quantification of GSDMD pore dynamics.

By accumulating the flare events in a representative cell over time into a single image, it was shown that flares did not have a simple spatial preference but occurred across the plasma membrane. Thus, the kinetics as well as the spatial extent of the calcium flares were analyzed using a verified subset. On average, calcium flares maximized in 0.35 minutes, and pores were open for an average of 1.55 minutes, reaching a mean diameter of ˜10 μm (n=21 pores). The pore closing was well-approximated by a kinetic inhibitory response from cumulative calcium; it was estimated that pore closing displayed a cooperative coefficient of 0.73 (mean of n=21; R²=0.996). Interestingly, the same pore could re-open in-place in the live-cell membrane, and ˜57% of the pores persisted after the mean open time and reopened. Faster calcium flares with an average t_{1/2} of 3.5 seconds could be found using higher frequency imaging. Image analysis showed that optogenetically-activated calcium signals emanated largely from circular pores. This evolved from a single spark in the center to a ring of high flux with the propagation of the calcium flare. Occluded, semi-circular flares were observed when a pore opened near the edge of the cell. Otherwise, asymmetrical events and catastrophic membrane leakage were not observed, indicating that pores did not close via disassembly. Within these geometric limits, the average circularity (minor/major radii ratio) of the flares was 0.75. Thus, in live-cell membranes, GSDMD pores likely opened and closed neatly as ellipsoidal, intact oligomer pores.

### Example 4: GSDMD Pore Dynamics is Phosphoinositide-Dependent

It was surmised that the dynamics of these oligomeric pores may stem from the thermodynamic influences of the lipid bilayer environment. To study the role of the bilayer, in vitro electrophysiology was employed (FIG. 3) to examine the kinetics of reconstituted GSDMD pore in a free-standing membrane bilayer. Following the formation of a stable phospholipid bilayer (POPE/POPC), all controls (bilayer-only, addition of either recombinant GSDMD or caspase-1 alone) showed no change in micro-current recordings (n=20 bilayers each, FIG. 4). However, upon the addition of activated GSDMD (mixture with caspase-1 to cleave the C-terminal autoinhibition domain), minute current fluctuations were detected indicating membrane-protein interactions that preceded pore formation (n=20 bilayers, FIG. 4, boxed region). On average, these micro-currents occurred 27.2±2.7 minutes after protein introduction. Shortly thereafter, a single pore ion flow was observed characterized by macro-currents. Strikingly, the macro-current traces also revealed that the recombinant pores closed and opened repeatedly in the model bilayers as was observed in live-cell membranes. Further analyses clearly contrasted the single pore events from those of unitary, single ion channels. The peak currents of single GSDMD pores appeared more varied compared to binary ON/OFF states typical of ion channels. The nonuniform peak currents was attributed to an “analog” character of these pores, whose oligomeric nature means that both the maximum pore size and the pore supra-structure are dynamic. The histogram of peak currents showed a broad distribution with a mean current of 32 pA. A largely uniform dwell time distribution indicated little preference in how long they remained open within the time window; an open probability of ˜0.2 indicated a slight preference for the closed state. A ramp protocol showed dual rectification, indicating these pores were not inherently ion-selective. Thus, the active GSDMD pores in artificial membranes spontaneously opened and closed, consistent with the above observation of calcium flares in live cells.

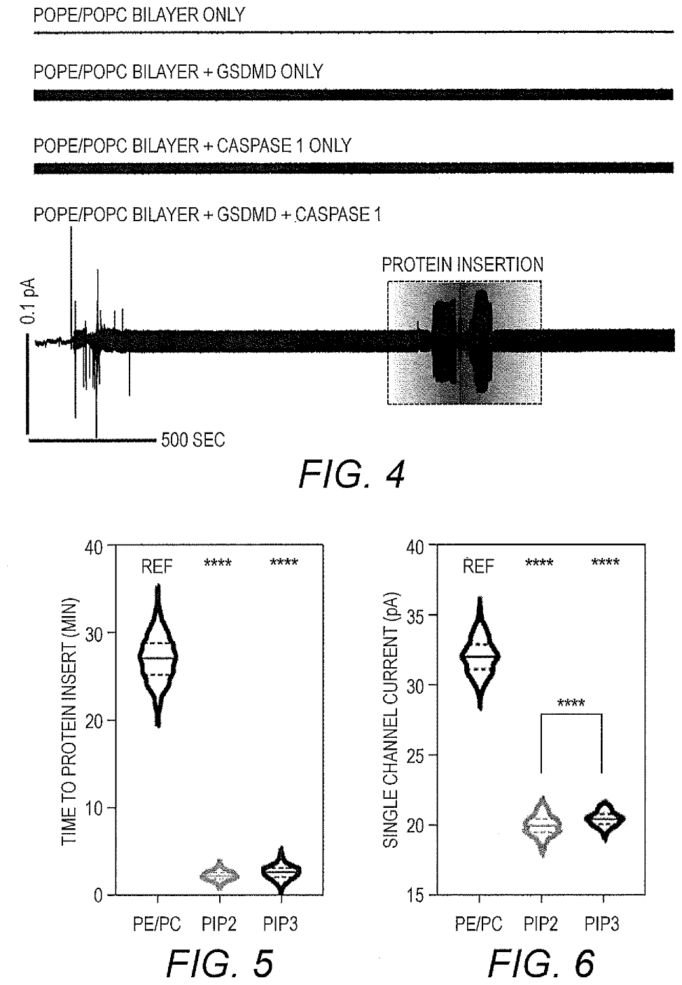

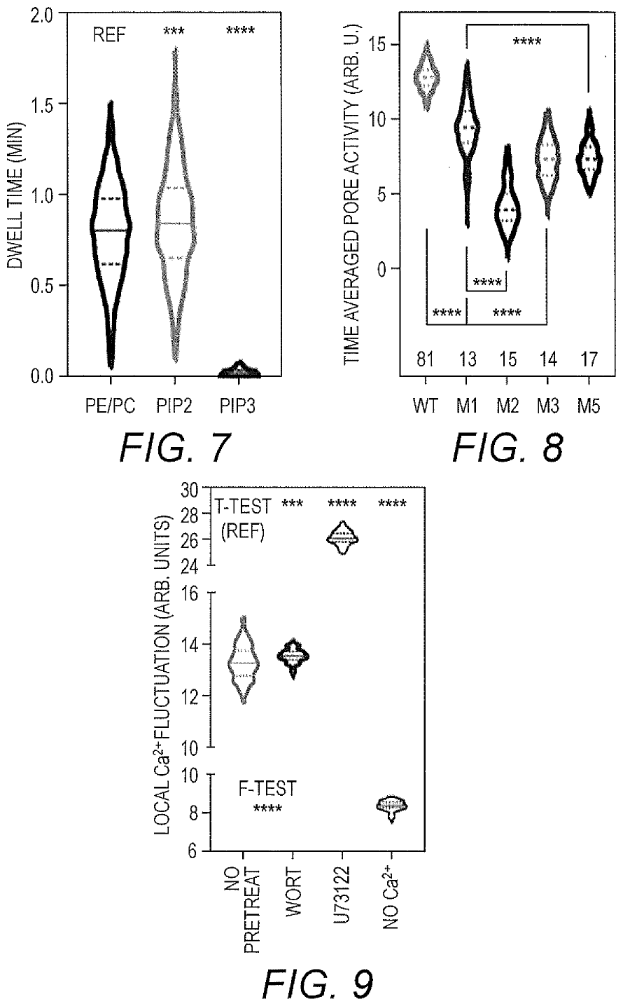

The observed kinetics of the live-cell pores are compatible with local, calcium-dependent modification of the membrane lipid composition. This would in turn create the thermodynamic conditions necessary for GSDMD pore closure as a negative feedback response shortly after opening. Importantly, the above dynamics were observed in a membrane without phosphoinositide, which was presumed to be required for GSDMD pores (Mulvihill et al. (2018) EMBO J. 37: e98321). As such, was it determined whether the role of phosphoinositide is instead to control GSDMD dynamics. Phospholipase C (PLC) and phosphoinositide 3-kinase (PI3K) are two major calcium-dependent phosphoinositide-modifying enzymes acting on the substrate PtdIns(4,5)P₂ (PIP2), present in the inner leaflet of the plasmalemma membrane. Specific PLC activity requires calcium and is further activated via transient calcium entry; PI3K is directly activated by calcium-calmodulin. Under calcium stimulation, the enzymes PLC PI3K and promote the relative accumulation of diacylglycerol (DAG) and phosphatidylinositol triphosphate (PtdIns (3,4,5) P3, or PIP3), respectively. Therefore, the reconstituted bilayer models were employed to examine the consequences of shifting membrane PIP2 content to either PIP3 or DAG. The introduction of PIP2 (10%) to the POPE/POPC baseline bilayer composition decreased the time to observe protein-membrane interaction significantly to 2.4±0.5 minutes (FIG. 5); PIP2 inclusion also significantly reduced the mean current of these pores to 20 pA (compared to 32 pA in baseline bilayers). The presence of PIP2 made the pores highly dynamic, leading to a significant 57-fold increase in open/close events. The current histogram suggested that PIP2 addition reduced the diversity of pore conformations (FIG. 6). However, PIP2 addition did not affect the open probability of these pores and the dwell time was similarly distributed compared to the baseline phospholipid bilayer (FIG. 7). Overall, PIP2 inclusion directly enhanced the dynamics of GSDMD pores. Of note, the inner mitochondrion membrane lipid cardiolipin (CL), another lipid target of GSDMD, also supported a pore dynamics profile similar to that of PIP2.

The equilibrium condition where all PIP2 had been metabolized by PLC was subsequently examined by substituting DAG for PIP2 in the lipid mixture while maintaining the fatty acid chain length (di-oleoyl) and relative concentration (10%). Despite observing micro-currents that signify protein-membrane interaction, no single pore activity was detected (n=15 bilayers) to quantify any macroscopic current. When PIP2 was replaced with PIP3, GSDMD protein-membrane interaction remained as rapid as with PIP2 (FIG. 5). The presence of PIP3, however, caused a rapid, cumulative increase in the macroscopic current that obscured clear, single pore open/close events (n=20 bilayers). The few single events captured showed similar mean current (20.5 pA, FIG. 6) but displayed a different conformational landscape and were significantly shorter-lived. Thus, PIP3 content kept GSDMD pores predominantly in the open state while in contrast, DAG content induced the closed state. Notably, phosphoinositide content had little effect on the rectification behavior and hence pore selectivity.

To explore the structural basis of the phosphoinositide control, two putative phosphoinositide interaction sites were altered and their contribution to GSDMD pore dynamics was examined. A three-dimensional structure alignment comparison between mGSDMA3 (PDB: 6CB8) and hGSDMD (PDB: 6N90) (Ruan et al. (2018) Nature 557:62-67; Liu et al. (2019) Immunity 51:43-49) identified two regions (I) R42/K43 and (II) K51/R53/K55 in hGSDMD as potential sites where membrane-inserted monomers may access the phosphoinositide headgroups; these sites were also occluded by the autoinhibitory C-terminal domain. Altering either charge or hydrogen-bonding potential, PhoDer mutants were prepared according to conventional methods and expressed in live Hela cells. These mutants included m1: K51Q/R53Q/K55Q (SEQ ID NO:34); m2: K51E/R53E/K55E (SEQ ID NO:35); m3: R42Q/K43Q (SEQ ID NO:36); m4: R42E/K43E (SEQ ID NO:37); m5: R42Q/K43Q/K51Q/R53Q/K55Q (SEQ ID NO:38) and m6: R42E/K43E/K51E/R53E/K55E (SEQ ID NO: 39). Calcium biosensing and automated flare quantifications were examined in the resulting cells. While these mutants were still capable of insertion and pore formation and thus showed calcium responses, they displayed significantly reduced pore opening/closing dynamics (FIG. 8). It also seemed possible to remove the saturating calcium response while still maintaining repeated open/close events. Charge-altering mutations at other putative lipid-binding sites could similarly change pore dynamics. Mutations at the well-conserved N-terminal region (i.e., R7E/R10E/R11E) retained clear flares but showed much-reduced calcium magnitude, while two mutants at another helical region (i.e., R137E and R151E/R153E) diverged, either encumbering or abrogating the calcium response. Thus, lipid-binding sites from at least three regions on the GSDMD N-terminal domain contribute to pore dynamics and can be specifically utilized to tune these dynamics.

### Example 5: Calcium-Driven Phosphoinositide Metabolism Regulates GSDMD Pore Dynamics and Cytokine Release

The lipid PI3K/PLC enzymes were subsequently perturbed to investigate whether these enzymes are actively used by living cells to tune and control GSDMD pore dynamics. Inhibition of PLC with U73122 (10 μM) prior to PhoDer activation caused a steep, cumulative rise in intracellular calcium in all cells (n=72 biologically independent cells) where individual flares were rarely observable. The U73122 pretreated cells rapidly reached the saturation and lytic stage. In contrast, PhoDer activation in cells pretreated with the pan-PI3K inhibitor wortmannin (10 μM) showed a significant delay in calcium progression across many cells (n=39 biologically independent cells). The delay of onset was further accompanied by significant changes in calcium flares dynamics. Analysis of these flares using the above fluctuation contrast imaging showed that pores under wortmannin pretreatment closed slowly and displayed a lower cooperative coefficient of 0.30 (mean of n=27 pores; R²=0.998). They reached a maximal calcium flux in 0.60 minutes, approximately twice that shown by pores on the untreated live-cell membrane. While the spatial extent these flares reached was not significantly different (12 μm, n=27), flares in wortmannin-treated cells showed less temporal alignment and a longer effective average opening time of 3.30 minutes; subsequent reopening was also more delayed and spread out in time. Pixel-wise calcium fluctuations were further used to compare the local pore activity across different treatments. U73122 pretreated cells displayed the highest activity per locale compared to control, non-treated cells. Wortmannin pretreated cells showed a comparable mean activity per locale as non-pretreated cells, but with significantly less variability (FIG. 9). Low, basal pixel-wise calcium activity was observed when extracellular calcium was withheld (ANOVA p<0.0001; FIG. 9). Together, these data showed that calcium-driven phosphoinositide composition shift significantly altered GSDMD pore dynamics.

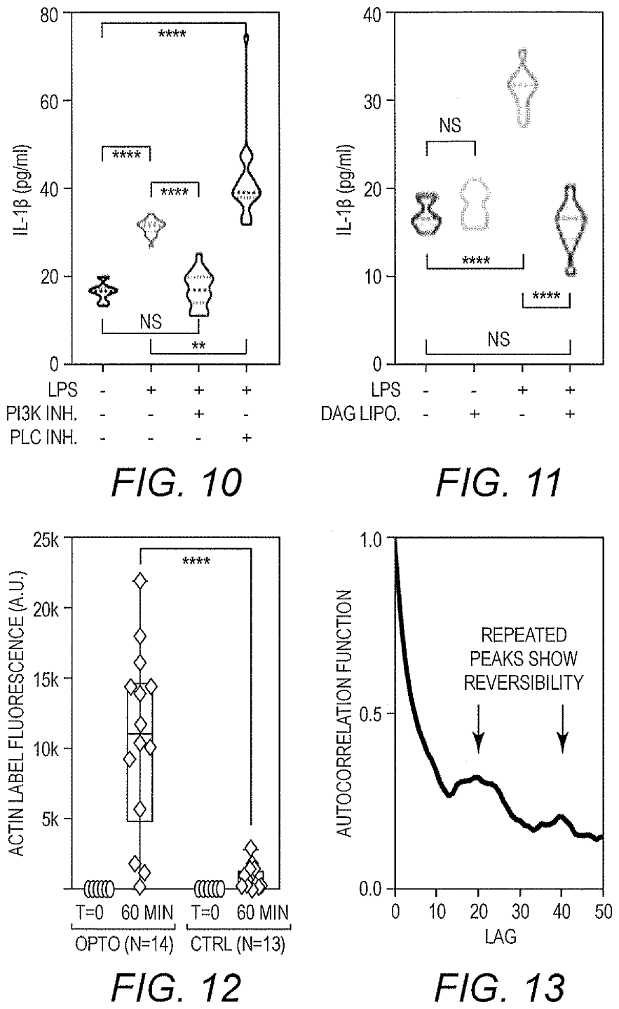

Previous studies showed that gasdermins could be irreversibly inhibited (Hu et al. (2020) Nat. Immunol. 21:736-745; Humphries et al. (2020) Science 369:1633-1637). The data suggest that modulating GSDMD dynamics through phosphoinositide feedback reversibly controls pore function and in turn, the release of inflammatory cytokines. Thus, the effects of regulating plasma membrane phosphoinositide dynamics on the release of pro-inflammatory IL-18 in LPS-stimulated mouse BMDM were examined. Consistent with the above observations, IL-1β release 5 minutes after LPS exposure was significantly dampened by wortmannin and vice versa, enhanced by U73122 pretreatment (FIG. 10). Following LPS-priming and nigericin-induced NLRP3 inflammasomal activation of GSDMD, a significant reduction of IL-16 release with PI3K inhibition was also observed. The effects of directly introducing phospholipids were also examined to alter live-cell inflammatory response. The results of this analysis indicate that dioleoylglycerol-rich liposomes treatment significantly suppressed both the short-term LPS-induced (FIG. 11) as well as the more prolonged nigericin-induced IL-1β release in BMDM. To probe the network integrity demanded by the GSDMD-calcium signaling, the PI3K/PLC balance was further manipulated through activators. While the PLC activator m-3m3FBS (Bae et al. (2003) Mol. Pharmacol. 63:1043-50) inhibited flare occurrence for a short time, its activation of other calcium signal (Krjukova et al. (2004) Br. J. Pharma. 143:3-7) meant that, unlike PI3K inhibition, it did not limit LPS-stimulated IL-1β release. The peptidic PI3K activator 740 Y-P (Williams & Doherty (1999) Mol. Cell. Neurosci. 13:272-280) also failed to elicit meaningful differences in calcium or cytokine release responses. These results demonstrate the highly specific and localized nature of the GSDMD-calcium circuit, further emphasizing the utility of the inhibitors described above. In aggregate, these data show that the calcium-influx driven phosphoinositide metabolism is a component of the GSDMD pore. PI3K/PLC enzymes form a major signaling circuit in regulating oligomeric Gasdermin D pore activity, through which cytokine release and hence the strength of the downstream inflammatory signals can be modulated.

### Example 6: Modulating Molecular Transport of a Cell

Using the PhoDer protein, it was determined whether a large molecule, i.e., an impermeable dye, fluorescent protein or an IgG antibody, could be transported into or out of a living cell via light-inducible activation of the PhoDer construct.

GFP Efflux. Cells expressing GFP were transfected with the PhoDer protein and illuminated intermittently for 1 hour. Over time the gasdermin pore grew larger and GFP fluorescence was captured on a fluorescence microscope using a 40× objective. This analysis showed that as the size of the gasdermin pore increased, GFP left the cell, as evident by a loss of intracellular fluorescence.

Phalloidin Influx. Phalloidin is a cell impermeable dye used in the labeling of F-actin. Cells were transfected with the PhoDer protein and contacted with phalloidin in the cell medium. The cells were subsequently illuminated and permeation of the phalloidin was monitored. This analysis showed that blue-light activation allowed phalloidin to permeate and bind F-actin (FIG. 12). Notably, stress fibers were clearly labeled after blue-light activation of the cells. In contrast, control cells expressing PhoDer protein, but not exposed to blue-light, did not exhibit any labeling of F-actin. Calcium imaging showed that the cells did not lyse but remained adherent and intact for a long period of type. As such, control gasdermin pore formation was capable of mediating cargo delivery in the absence of cell death.

Antibody Delivery. Hela cells expressing PhoDer were placed in medium containing a fluorescently tagged anti-tubulin monoclonal IgG antibody (˜150 kDa, ˜14 nm in size). Before exposure to light, antibody fluorescence was evident in the medium. Upon selective permeabilization via a short (about 5 minute) blue-light activation, the fluorescent antibody was delivered into the interior of the cell. Because the antibody was not endocytosed, it did not present as fluorescent vesicles, but was localized to tubulin. After washing the cells to remove any extracellular antibody, the cells were observed to be intact and adherent for at least 24 hours. Notably, the pores closed soon after opening. Super-resolution imaging of the cells was carried out using STORM (Stochastic Optical Reconstruction Microscopy). This analysis showed fluorescence in thin clusters and tubulin bundles. Advantageously, the cells remained intact and alive thereby allowing for analysis of cytoskeletal tubulin and nuclear tubulin. Notably, conventional fixed cell staining does not allow for nuclear tubulin analysis. Given that cytoskeletal and nuclear tubulin have distinct functions apart from their structural roles in forming microtubules, delivery of the anti-tubulin antibody via the present invention allows for further subcellular analysis of tubulin in living cells.

Reversible Optogenetics. HeLa cells expressing a modified gasdermin protein of the invention (SEQ ID NO: 40) were subjected to cyclic activation. Whereas gasdermin lacking a photoactivatable linker has no ability to reverse and will kill the cell typically in 30 minutes or less, activation of reversible optogenetic gasdermin allow cells to stay alive for 2 hours or longer. Furthermore, upon closer examination, the cyclic activation pattern can clearly be detected in the response of these cells via autocorrelation function (FIG. 13). The “imprinting” of this cyclic pattern into the cellular response can only be possible if the optogenetic gasdermin protein is reversible.

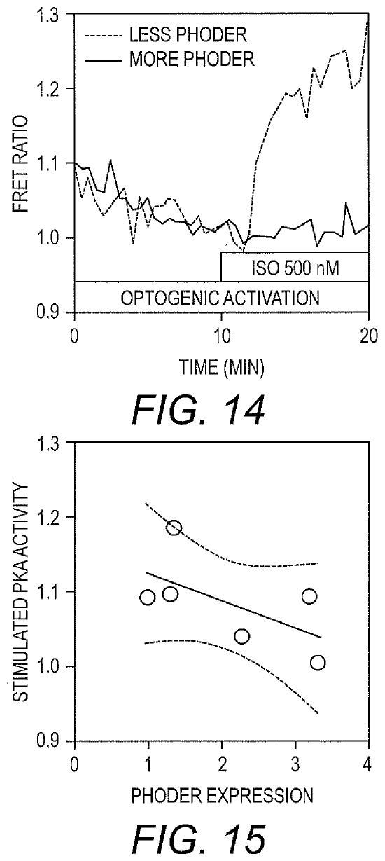

Nanobody Delivery. Nanobodies have been developed that inhibit beta2-adrengeric receptor (b2AR) by binding the cytosolic loops of the receptor. However, these nanobodies have not been therapeutically useful because nanobodies are large proteins that cannot diffuse across the cellular membrane into the cytosol to affect cell signaling and have a therapeutic effect. To demonstrate the use of the optogenetic gasdermin protein of the invention to deliver a nanobody, HeLa cells expressing PhoDer were exposed to purified nanobodies in the extracellular milieu and activated with blue-light. After 10 minutes, the cells were challenged with isoproterenol (ISO, 500 nM), a well-known b2AR agonist that causes activation of both the receptor and its downstream kinase PKA. Thus, PKA activity was monitored during treatment. In cells expressing less PhoDer, a strong and fast stimulated PKA response was observed, indicating a lack of b2AR inhibition. By comparison, in cells expressing a higher level of PhoDer, there is a clear inhibition of ISO-stimulated responses (FIG. 14). This effect is clearly demonstrated across many cells (FIG. 15). Accordingly, a modified gasdermin protein of this invention finds use in the delivery of a nanobody into the cytosol of cells in order to achieve a therapeutic effect.

What is claimed is:

1. A modified gasdermin protein comprising a photoactivatable linker inserted between the N-terminal domain and C-terminal domain of a gasdermin protein, wherein the photoactivatable linker dimerizes or dissociates upon illumination.

2. The modified gasdermin protein of claim 1, wherein the photoactivatable linker is inserted at an endogenous protease cleavage site of the gasdermin protein.

3. The modified gasdermin protein of claim 1, wherein the photoactivatable linker is an optogenetic dimerization protein selected from the group of Vivid, cryptochrome, N-terminal domain of cryptochrome-interacting basic-helix-loop-helix protein 1, phytochrome, phytochrome interacting factor, UV-B photoreceptor, Flavin-binding Kelch repeat F-box 1, GIGANTEA, TULIPS, Dronpa, iLID, AsLOV variant, and combinations thereof.

4. The modified gasdermin protein of claim 1, wherein the photoactivatable linker is a photocleavable protein that dissociates into at least two fragments or releases one end of a loop insertion upon illumination.

5. The modified gasdermin protein of claim 4, wherein the photocleavable protein is PhoCle0.1, PhoCle0.2, PhoCle0.3, PhoCle0.4, PhoCle0.5, PhoCle0.6, PhoCle0.7, cpPhoCle, PhoCl2c or PhoCl2f.

6. The modified gasdermin protein of claim 4, wherein the photocleavable protein comprises the amino acid sequence of SEQ ID NO:23, SEQ ID NO:24, SEQ ID NO:25, SEQ ID NO: 26, SEQ ID NO:27, SEQ ID NO:28, SEQ ID NO:29, SEQ ID NO: 30, or SEQ ID NO:31.

7. The modified gasdermin protein of claim 6, wherein the photocleavable protein is cleaved by illumination with light having a wavelength of about 400 nm to 450 nm.

8. The modified gasdermin protein of claim 1 wherein the gasdermin protein is gasdermin A, gasdermin B, gasdermin C, gasdermin D, gasdermin E, pejvakin, or a fragment thereof.

9. The modified gasdermin protein of claim 8, wherein the gasdermin protein is selected from the group consisting of human gasdermin D, human gasdermin B and mouse gasdermin A3.

10. A modified gasdermin protein of claim 1 comprising the amino acid sequence of SEQ ID NO: 33, SEQ ID NO: 34, SEQ ID NO:35, SEQ ID NO: 36, SEQ ID NO: 37, SEQ ID NO: 38, SEQ ID NO:39 or SEQ ID NO: 40.

11. A recombinant nucleic acid encoding the modified gasdermin protein of claim 1.

12. A recombinant vector comprising the nucleic acid of claim 11.

13. A recombinant host cell comprising the modified gasdermin protein of claim 1.

14. A method of modulating gasdermin pore formation in a cell comprising inserting a photoactivatable linker between the N-terminal domain and C-terminal domain of a gasdermin protein of a cell and illuminating the cell with light having a wavelength suitable to activate the photoactivatable linker, thereby modulating gasdermin pore formation in the cell.

15. A method of treating a disease or condition in a subject wherein gasdermin pore formation in a cell of the subject confers a benefit inserting comprising a photoactivatable linker between the N-terminal domain and C-terminal domain of a gasdermin protein of a cell and illuminating the cell with light having a wavelength suitable to activate the photoactivatable linker, thereby modulating gasdermin pore formation in the subject and treating the disease or condition.

16. A method of facilitating transport of a therapeutic agent into a cell comprising inserting a photoactivatable linker between the N-terminal domain and C-terminal domain of a gasdermin protein of a cell; contacting the cell with a therapeutic agent; and illuminating the cell with light having a wavelength suitable to activate the photoactivatable linker and modulate gasdermin pore formation thereby facilitating transport of the therapeutic agent into the cell.

17. A method of facilitating treatment of a disease or condition in a subject comprising inserting a photoactivatable linker between the N-terminal domain and C-terminal domain of a gasdermin protein of a subject; administering to the subject a therapeutic agent for treating a disease or condition; and exposing the subject to light having a wavelength suitable to activate the photoactivatable linker and modulate gasdermin pore formation so that transport of the therapeutic agent is enhanced and treatment of the disease or condition is facilitated.


SEQUENCE LISTING (40 sequences)

SEQ ID NO: 1 - protein, 445 aa, Homo sapiens
MTMFENVTRALARQLNPRGDLTPLDSLIDFKRFHPFCLVLRKRKSTLFWGARYVRTDYTLLDVLEPGSSPSDPTDTGNFGFKNMLDTRVEGDVDVPKTVKVKGTAGLSQNSTLEVQTLSVAPKALETVQERKLAADHPFLKEMQDQGENLYVVMEVVETVQEVTLERAGKAEACFSLPFFAPLGLQGSINHKEAVTIPKGCVLAFRVRQLMVKGKDEWDIPHICNDNMQTFPPGEKSGEEKVILIQASDVGDVHEGFRTLKEEVQRETQQVEKLSRVGQSSLLSSLSKLLGKKKELQDLELALEGALDKGHEVTLEALPKDVLLSKEAVGAILYFVGALTELSEAQQKLLVKSMEKKILPVQLKLVESTMEQNFLLDKEGVFPLQPELLSSLGDEELTLTEALVGLSGLEVQRSGPQYMWDPDTLPRLCALYAGLSLLQQLTKAS

SEQ ID NO: 2 - protein, 464 aa, Mus musculus
MPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEMHEDFKTLKEEVQRETQEVEKLSPVGRSSLLTSLSHLLGKKKELQDLEQKLEGALDKGQKVTLEALPKDVLLSKDAMDAILYFLGALTELTEEQLKILVKSLEKKILPVQLKLVESTLEQNFLQDKEGVFPLQPDLLSSLGEEELTLTEALVGLSGLEVQRSGPQYAWDPDTRHNLCALYAGLSLLHLLSRKSNALTYCALS

SEQ ID NO: 3 - protein, 446 aa, Rattus norvegicus
MTMFENVTRALARQLNPRGDLTPLDSLIDFKCFHPFCLVLRKRKSTLFWGARYVRTDYTLLDLLEPGNTPSDPTDGGNFSFKNMLDARVEGDVDVPKTVKVKGTAGLSQSSTLEVQTLSVAPKALESLHKERKLSADHPFLKEMWERGENLYVVMEVVETVQEVTLERAGKAEGCFSLPFFAPLGLQGSLNHKEAVTIPKGCVLAYRVRQLMVNGKDEWDIPHVYNDSMQTFPPGEKPGEGKFILIQASDVGEMHDDFKTLKEEVRRETREIEKLSQVGRSSLLTSLRHLLGKKKELQDLEHTLEGALDKGHEVTLEALPKDVLLSKDAMDAILYFLGALTVLSEAQQKLLVKSLEKEILPLQLKLVESTLEQNFLQDKEGVFPLRPDLLSSLGEEELTLTEALVGLSGLEVQRSGPQYTWDPDTLPHLCALYAGLSLLQLLSMGS

SEQ ID NO: 4 - protein, 411 aa, Homo sapiens
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMSAGLDIHFRGKTKSFPEGKSLGSEDSRNMKEKLEDMESVLKDLTEEKRKDVLNSLAKCLGKEDIRQDLEQRVSEVLISGELHMEDPDKPLLSSLFNAAGVLVEARAKAILDFLDALLELSEEQQFVAEALEKGTLPLLKDQVKSVMEQNWDELASSPPDMDYDPEARILCALYVVVSILLELAEGPTSVSS

SEQ ID NO: 5 - protein, 443 aa, Bos taurus
MPTVFESISRAVVKEIDTSGETIAVRSLSDADKFHCSYLVKKRRRFFGYQYDKTNLTLKDILECEVPFDMVVPELQGDLPDSGIKPASAALPEDSLPLSHLGNPHNKGQGDNFEILDVVDSKGSLAVKFHPQMTFKIAFHIFQEKNIKLEKSEIPQEFLDSLKNKKLKKELPPSFQSIQAKREDLYLVTETLKTKRTETLKYETQFGFQILLKMFGFQCKHKHQKEVTITPEKVLAYRVKQLVFPSAERMDICFLDKTRSFPEEKDGGSSWLGKSLNLENFRSIKERVQDMVRVLQDLTPEEQKEALNCFTKCLSSEEELQDLEQRVSEVQCSGELQMNSPANSLISSLFNAAGALIEARAETIWDVLDALMELSEYRQFVAEVLDKGELRPVKDKVESILEENRCEGPLDVSCDPEARTLCALYVVVSILLQLSEKPTSVSS

SEQ ID NO: 6 - protein, 508 aa, Homo sapiens
MPSMLERISKNLVKEIGSKDLTPVKYLLSATKLRQFVILRKKKDSRSSFWEQSDYVPVEFSLNDILEPSSSVLETVVTGPFHFSDIMIQKHKADMGVNVGIEVSVSGEASVDHGCSLEFQIVTIPSPNLEDFQKRKLLDPEPSFLKECRRRGDNLYVVTEAVELINNTVLYDSSSVNILGKIALWITYGKGQGQGESLRVKKKALTLQKGMVMAYKRKQLVIKEKAILISDDDEQRTFQDEYEISEMVGYCAARSEGLLPSFHTISPTLFNASSNDMKLKPELFLTQQFLSGHLPKYEQVHILPVGRIEEPFWQNFKHLQEEVFQKIKTLAQLSKDVQDVMFYSILAMLRDRGALQDLMNMLELDSSGHLDGPGGAILKKLQQDSNHAWFNPKDPILYLLEAIMVLSDFQHDLLACSMEKRILLQQQELVRSILEPNFRYPWSIPFTLKPELLAPLQSEGLAITYGLLEECGLRMELDNPRSTWDVEAKMPLSALYGTLSLLQQLAEA

SEQ ID NO: 7 - protein, 468 aa, Mus musculus
MSYTFDWLSKDVVKKLQGRDLRPVKCLSDATKFCLFNILQETSSRLALKTEYIPVGFTLLHLLEPNIPVPEPEVSAPIPLKHTISQKLKADLDVETIAGGEAGFVKSCGYDIEVQSKSIPNPKLESLQNRKLLDQLPTFMKTCWKDGKNLYVVTEAYEVTKDTVLEGTSNSKFAIKGIINQLVKVGGSGQWQTEKTDSIPIQKGSVLAYKKQQLVIEDNTCVILTSANTKKKMTFPMRFVGMSGHLRYQDLVIETGSWINDIDPIGTIKEPTHLDFMCLQNEVSEQTRLLAELSKDVQEVVFSSFLHMLCDRDVLYDLMKMLELNQLGHMDGPGGKILDELRKDSSLSWINLKDLILYLLQALMVLSDTQLCLLALSVEMRLLPHQVELVKSILQPNFKYPWNIPFTLQPQLLAPLQGEGLAITYELLEECGLKMELNNPRSTWDLEAKMPLSALYGSLSFLQQLSEA

SEQ ID NO: 8 - protein, 467 aa, Rattus norvegicus
MLYTFDQVSKDVVKKLQGKDLRPVRCLSDATKFRQFDILQKTPQSLFFKSEDTPVGYSLLQILEPNFPVPETEVSAPMPLKHITSQKWKADVDVKATIADGGASAEFVQSCGYDIEVQSRSIPDSKLESLQNRKLLDKKLSFVTDCQMGRNNLYVVTEVFEVTKDTVVQGSSSIDLSGKALVSQLVKGEAQGQWQRETTDLVPIPKGAVLAYKKKQLVIENNTCAILLSANAKKKTFPGIFNFGMSSRSQTMEIGSWIDYIPPIGRIEEPVHLDFKYLEKEVFLRKEQLAMLSKDVQDVVFSNLLPMLSDSDVLFDLINMLELDQLGHMDGPAGLILDELRKNSSTPWIDLKGLILYLLQALMVLSDTQLDLLAQSMEMRILLQQRELVRSILEPNFKYPWNIPFTLQPQLLAPLQGEGLAITYELLKGCGLKMEPNSPRSTWDLEAKMPLSALYGILSCLQQLVEA

SEQ ID NO: 9 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 10 - protein, 487 aa, Mus musculus
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSLLSDGIDEEELIEAADFQGLYAEVKACSSELESLEMELRQQILVNIGKILQDQPSMEALEASLGQGLCSGGQVEPLDGPAGCILECLVLDSGELVPELAAPIFYLLGALAVLSETQQQLLAKALETTVLSKQLELVKHVLEQSTPWQEQSSVSLPTVLLGDCWDEKNPTWVLLEECGLRLQVESPQVHWEPTSLIPTSALYASLFLLSSLGQKPC

SEQ ID NO: 11 - protein, 496 aa, Homo sapiens
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPDAAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHS

SEQ ID NO: 12 - protein, 512 aa, Mus musculus
MFAKATRNFLKEVDAGGDLISVSHLNDSDKLQLLSLVTKKKRYWCWQRPKYQILSATLEDVLTEGHCLSPVVVESDFVKYESKCENHKSGAIGTVVGKVKLNVGGKGVVESHSSFGTLRKQEVDVQQLIQDAVKRTVNMDNLVLQQVLESRNEVLCVLTQKIMTTQKCVISEHVQSEETCGGMVGIQTKTIQVSATEDGTVTTDTNVVLEIPAATTIAYGIMELFVKQDGQFEFCLLQGKHGGFEHERKLDSVYLDPLAYREFAFLDMLDGGQGISSQDGPLRVVKQATLHLERSFHPFAVLPAQQQRALFCVLQKILFDEELLRALEQVCDDVAGGLWSSQAVLAMEELTDSQQQDLTAFLQLVGYRIQGEHPGPQDEVSNQKLFATAYFLVSALAEMPDNATVFLGTCCKLHVISSLCCLLHALSDDSVCDFHNPTLAPLRDTERFGIVQRLFASADIALERMQFSAKATILKDSCIFPLILHITLSGLSTLSKEHEEELCQSGHATGQD

SEQ ID NO: 13 - protein, 507 aa, Rattus norvegicus
MFAKATRNFLKEVDAGGDLISVSHLNDSDKLQLLSLVTKKKRYWCWQRPKYQFLSATLGDVLTEGHCLSPVVVESDFVKYESKCENHKSGTIGTVMGKVKLNVGGKGLVESQSSFGTLRKQEVDVQQLIQDAVGRTVNLDSLVLQQVLESRNEVLCVLTQKIVTTQKCVISEHVQSEETCGGMVGIQTKIVQVPHLSMEDVSHGRLRRAVFGSGLREAWRKSTVMPEFCLLQGKHGGFEQERKVDSVYLDPLAYREFAFLDMPDGGQGISSQDGPLRVLKQATLHLERSFRSFAVLPAQQQMALFCILQKILFDEELLRALEQVCDDVAGGLWSSQAILAMEELTGSQQQDLTAFLKLVGYRVQGEHPGPQDEVSNQKLFATAYFLVSALAEMPDNATVFLGTCCKLHVIPSLCHLLHALSDDSVCDFQDPTLAPLRDTERFGIVQRLFASADIALERMQFSAKATILKDSCMFPLILHITLSGLYTLSKEHEEHLFQSEVHATSQD

SEQ ID NO: 14 - protein, 352 aa, Homo sapiens
MFAAATKSFVKQVGDGGRLVPVPSLSEADKYQPLSLVVKKKRCFLFPRYKFTSTPFTLKDILLGDREISAGISSYQLLNYEDESDVSLYGRRGNHIVNDVGINVAGSDSIAVKASFGIVTKHEVEVSTLLKEITTRKINFDHSLIRQSRSSRKAVLCVVMESIRTTRQCSLSVHAGIRGEAMRFHFMDEQNPKGRDKAIVFPAHTTIAFSVFELFIYLDGAFDLCVTSVSKGGFEREETATFALLYRLRNILFERNRRVMDVISRSQLYLDDLFSDYYDKPLSMTDISLKEGTHIRVNLLNHNIPKGPCILCGMGNFKRETVYGCFQCSVDGQKYVRLHAVPCFDIWHKRMK

SEQ ID NO: 15 - protein, 352 aa, Bos taurus
MFAAATKSFVKQVGDGGRLVPVPSLSEADKYQPLSLVVKKKRCFLFPRYKFTSTPFTLKDILLGNREISAGISSYQLLNYEDESDVSLYGRRGNHIVSDVGINVTGSDSIAVKASFGVVTKHEVEVSTLLKEITTRKINFDHSLIRQSRSSRKAVLCVVMESIRTTRQCSLSVHAGIRGEAMRFHFMDEQNPKGRDKAIVFPAHTTIAFSVFELFIYLDGAFDLCVTSVSKGGFEREETATFALLYRLRNILFERNRRVMDAISRSQLYLDDLFSDYYDKPLSMSDISLKEGTHIRVNLLNHNIPKGPCILCGMGNFKRETVYGCFQCSVDGQKYVRLHAVPCFDIWHKRMK

SEQ ID NO: 16 - protein, 352 aa, Mus musculus
MFAAATKSFVKQVGDGGRLVPVPSLSEADKYQPLSLVVKKKRCFLFPRCKFTSTPFTLKDILLGDREISAGISSYQLLNYEDESDVSLYGRRSNHIVNDVGINVTGSDSIAVKASFGVVTKHEVEVSTLLKEITARKINFDHSLIRQSRSSRKAVLCVVMESIRTTRQCSLSVHAGIRGEAMRFHFMDEQNPKGREKAIVFPAHTTIAFSVFELFIYLDGAFDICVTSVSKGGFEREETTTFAMFYRLRNILFERNRRVMDAISRSQLYLDDLFSDFYDKPLSMTDISLKEGTHIRVNLLNHNIPKGPCILCGMGNLKRETVYGCFQCSVDGVKYVRLHAVPCFDIWHKRMK

SEQ ID NO: 17 - protein, 7 aa, Homo sapiens
QTFPPGE

SEQ ID NO: 18 - protein, 7 aa, Homo sapiens
DVLNSLA

SEQ ID NO: 19 - protein, 7 aa, Homo sapiens
DELDSGL

SEQ ID NO: 20 - protein, 7 aa, Homo sapiens
SSNDMKL

SEQ ID NO: 21 - protein, 7 aa, Homo sapiens
FLTDGVP

SEQ ID NO: 22 - protein, 7 aa, Homo sapiens
DMPDAAH

SEQ ID NO: 23 - protein, 243 aa, Artificial Sequence
  Synthetic polypeptide
SVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 24 - protein, 242 aa, Artificial Sequence
  Synthetic polypeptide
VIPDYFKQSFPEGYSWERSMTYEDGGICIATNDITMEEDSFINKIHFKGTNFPPNGPVMQKRTVGWEVSTEKMYVRDGVLKGDVKMKLLLKGGSHYRCDFRTTYKVKQKAVKLPDYHFVDHRIEILSHDKDYNKVKLYEHAVARNSTDSMDELYKGGSGGMVSKGEETIMSVIKPDMKIKLRMEGNVNGHAFVIEGEGSGKPFEGIQTIDLEVKEGAPLPFAYDILTTAFHYGNRVFTKYPR

SEQ ID NO: 25 - protein, 242 aa, Artificial Sequence
  Synthetic polypeptide
VIPDYFKQSFPEGYSWERSMTYEDGGICIATNDITMEEDSFINKIHFKGTNFPPNGPVMQKRTVGWEVSTEKMYVRDGVLKGDVKMKLLLKGGSHYRCDFRTTYKVKQKAVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 26 - protein, 242 aa, Artificial Sequence
  Synthetic polypeptide
VIPDYFKQSFPEGYSWERSMTYEDGGICIATNDITMEEDSFINKIHFKGTNFPPNGPVMQKRTVGWEVSTEKMYVRDGVLKGDVKMKLLLKGGSHYRCDYRTTYKVKQKAVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 27 - protein, 242 aa, Artificial Sequence
  Synthetic polypeptide
VIPDYFKQSFPEGYSWERSMTYEDGGICIATNDITMEEDSFINKIHFKGTNFPPNGPVMQKRTVGWEVSTEKMYVRDGVLKGDVKMKLLLKGGGHYRCDYRTTYKVKQKAVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 28 - protein, 242 aa, Artificial Sequence
  Synthetic polypeptide
VIPDYFKQSFPEGYSWERSMTYEDGGICIATNDITMEGDSFINKIHFKGTNFPPNGPVMQKRTVGWEASTEKMYVRDGVLKGDVKMKLLLKGGGHYRCDYRTTYKVKQKAVKLPDSHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 29 - protein, 242 aa, Artificial Sequence
  Synthetic polypeptide
VIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKAVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 30 - protein, 242 aa, Artificial Sequence
  Synthetic polypeptide
VIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 31 - protein, 236 aa, Artificial Sequence
  Synthetic polypeptide
VSKGEETITSVIKPDMKNKLRMEGNVNGHAFVIEGEGSGKPFEGIQTIDLEVKEGAPLPFAYDILTTAFHYGNRVFTKYPRSIPDYFKQSFPEGYSWERSMTYEDGGICIATNDITMEEDSFINKIHFKGTNFPPNGPVMQKRTVGWEVSTEKMYVRDGVLKGDVKMKLLLKGGSHYRCDFRTTYKVKQKAVKLPDYHFVDHRIEILSHDKDYNKVKLYEHAVARNSTDSMDELYK

SEQ ID NO: 32 - protein, 4 aa, Artificial Sequence
  Synthetic peptide
GGGS

SEQ ID NO: 33 - protein, 728 aa, Artificial Sequence
  Synthetic polypeptide
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGSVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 34 - protein, 728 aa, Artificial Sequence
  Synthetic polypeptide
MGSAFERVVRRVVQELDHGGEFIPVTSLQSSTGFQPYCLVVRKPSSSWFWQPQYQCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGSVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 35 - protein, 728 aa, Artificial Sequence
  Synthetic polypeptide
MGSAFERVVRRVVQELDHGGEFIPVTSLQSSTGFQPYCLVVRKPSSSWFWEPEYECVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGSVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 36 - protein, 728 aa, Artificial Sequence
  Synthetic polypeptide
MGSAFERVVRRVVQELDHGGEFIPVTSLQSSTGFQPYCLVVQQPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGSVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 37 - protein, 728 aa, Artificial Sequence
  Synthetic polypeptide
MGSAFERVVRRVVQELDHGGEFIPVTSLQSSTGFQPYCLVVEEPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGSVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 38 - protein, 728 aa, Artificial Sequence
  Synthetic polypeptide
MGSAFERVVRRVVQELDHGGEFIPVTSLQSSTGFQPYCLVVQQPSSSWFWQPQYQCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGSVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 39 - protein, 728 aa, Artificial Sequence
  Synthetic polypeptide
MGSAFERVVRRVVQELDHGGEFIPVTSLQSSTGFQPYCLVVEEPSSSWFWEPEYECVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGSVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 40 - protein, 634 aa, Artificial sequence
  Synthetic polypeptide
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNGGGGMLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGGGGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH